(19) United States
(12) Patent Application Publication
(10) Pub. No.: US 2015/0038500 A1
(43) Pub. Date: Feb. 5, 2015

(54) PHARMACEUTICAL COMPOSITION FOR PREVENTING AND TREATING OPHTHALMIC DISORDERS

(71) Applicant: CATHOLIC UNIVERSITY INDUSTRY-ACADEMIC COOPERATION FOUNDATION, Seoul (KR)

(72) Inventors: In-Beom Kim, Seongnam-si (KR); Muyan Kim, Seoul (KR); Sung Won Jung, Bucheon-si (KR); Eojin Jeong, Seoul (KR); Sooyeon Sim, Seoul (KR)

(21) Appl. No.: 14/359,624

(22) PCT Filed: Nov. 9, 2012
(86) PCT No.: PCT/KR2012/009481

§ 371 Date: Sep. 25, 2014

(30) Foreign Application Priority Data
Nov. 25, 2011 (KR) 10-2011-0124495

(51) Int. Cl. A61K 31/541; A01N 43/84

(52) U.S. Cl. CPC A61K 31/541; A01N 43/84
USPC 514/228.2; 544/58.2

(57) ABSTRACT

The invention relates to a pharmaceutical composition for preventing or treating an ophthalmologic disease comprising a compound of formula (1) as defined in the specification, or a pharmaceutically acceptable salt or isomer thereof as an active ingredient. Also, the invention relates to a composition for cleansing or preserving a contact lens comprising the above active ingredient.

### FIELD OF THE INVENTION

The present invention relates to a pharmaceutical composition for preventing or treating an ophthalmologic disease comprising a compound of formula (1) as defined in the specification, or a pharmaceutically acceptable salt or isomer thereof as an active ingredient. Also, the invention relates to a composition for cleansing or preserving a contact lens comprising the above active ingredient.

### BACKGROUND OF THE INVENTION

Eyes are an important sensory organ that receives most information necessary for living. An eye consists of an outer membrane, a middle membrane, an inner membrane and a refractive medium, and the outer membrane is comprised of a cornea which is a front surface covering a black pupil and a sclera which is connected thereafter, the middle membrane is comprised of an iris, a ciliary body and a choroid, and the inner membrane is comprised of a retina. A lens, vitreous body and aqueous humor fall under the refractive media. An eye's functional disorder or loss becomes one of the major factors degrading the quality of life, and maintaining healthy eyes becomes important because of aging, diseases, and other factors that may give bad influences on eye sight. Examples of ophthalmologic diseases include retina diseases including retinal degenerative disease and glaucoma, cataract, keratoconjunctival epithelial damage or corneal epithelial wound.

Retina-related ophthalmologic diseases may include retinal degenerative diseases including retinal degeneration, retinal pigment degeneration, retinal detachment, retinal tear, retinal ischemic disease and diabetic retinopathy, and glaucoma is a disease causing the loss of retinal ganglion cells and is closely related to the retina diseases. The retinal degenerative diseases are progressive diseases caused by environmental factors such as genetic or oxidative stress where the loss of eye sight occurs by the degeneration of photoreceptor cells, and in most cases, night blindness and a decrease in peripheral vision occur from an early stage of the diseases but the central vision is relatively preserved well and then weakened at a later stage. The glaucoma is a disease group consisting of several conditions showing various clinical findings and histopathological findings, and it shows conditions such as a change in optic disc, the injury of retinal ganglion cells and the resultant vision loss.

The cataract is an ophthalmologic disease in which vision gets blurry due to the opacification of the lens of an eyeball. The causes of the cataracts are very complicated, and systemic diseases such as diabetes, hyperparathyroidism, etc. have been reported to accelerate the progress of the cataracts but it is difficult to identify the causes of cataracts occurring in adults with no systemic diseases. In the latter cases, numerous factors including UV, heat, imbalance of hormones such as estrogen, relationship with smoking, etc. have been reported to be involved, but it is quite difficult to prove their influences.

Cornea-related ophthalmologic diseases may include keratoconjunctival epithelial damage or corneal epithelial wound. The keratoconjunctival epithelial damage is a defect in corneal epithelial cells constituting a corneal epithelial layer at the very surface layer of the cornea, and the corneal epithelial wound refers to a wound in a broad sense including injuries resulted from the tear, incision, or perforations of corneal epithelial tissues.

Currently, there are laser treatment, photocoagulation, cryotherapy, and photodynamic therapy as treatments for such ophthalmologic diseases. These treatments are all treatments based on surgery, and a drug-based treatment is still at a developing stage. The surgery-based treatments have a limit in that they are not applicable to all the patients, they have a low success rate and they cost a lot, so that they cause social and economic burdens. Most patients who cannot undergo surgery go blinded under such a circumstance that there is no currently available special treatment drug. Since such ophthalmologic diseases consistently increase while the life span of humans is being extended, it is urgent to develop an appropriate therapeutic agent.

Ophthalmologic disease therapeutic agents which are currently being developed are mostly steroids, MMP (matrix metalloproteinase) inhibitors, angiogenesis inhibitors, and antibodies against vasculogenesis growth factors. The present invention proposes a novel therapy via a drug besides the existing therapeutic methods for ophthalmologic diseases relying only on surgical operation.

Meanwhile, Korea patent laid-open No. 10-2009-0018593 provides a novel indole or indazole compound having necrosis-suppressive activity and a therapeutic agent for necrosis-related diseases comprising the same. However, while this patent discloses only necrosis-suppressive activity by the indole or indazole compounds, it discloses their relationship only with some necrosis-related diseases such as liver disease, neurodegenerative disease, etc., and it does not mention at all any possibilities of the compounds being applicable to treat ophthalmologic diseases.

### SUMMARY OF THE INVENTION

While the inventors were doing researches to provide compounds effective for preventing or treating ophthalmologic diseases, they have found that the compounds of Korea patent laid-open No. 10-2009-0018593 already known as a necrosis suppressor also show excellent effects for the prevention and treatment of ophthalmologic diseases, and thus completed the invention.

Accordingly, it is an object of the invention to provide a use of an indole or indazole derivative of formula (1) for preventing and treating an ophthalmologic disease,

Specifically, it is one object of the invention to provide a pharmaceutical composition for preventing and treating an ophthalmologic disease, comprising a compound of formula (1), or a pharmaceutically acceptable salt or isomer thereof. Also, it is another object of the invention to provide a method of treating an ophthalmologic disease, comprising administering to a subject a therapeutically effective amount of a compound of formula (1), or a pharmaceutically acceptable salt or isomer thereof. Also, it is another object to provide a use of a compound of formula (1), or a pharmaceutically acceptable salt or isomer thereof for use in the treatment of an ophthalmologic disease.

Preferably, the ophthalmologic disease includes cataract, glaucoma, retinal degeneration, retinal pigment degeneration, retinal detachment, retinal tear, retinal ischemic disease, diabetic retinal retinopathy, keratoconjunctival epithelial damage or corneal epithelial wound.

It is another object of the invention to provide a composition for cleansing or preserving a contact lens, comprising a compound of formula (1), or a pharmaceutically acceptable salt or isomer thereof. Also, it is another object of the invention to provide a method of cleansing or preserving a contact lens, using a compound of formula (1), or a pharmaceutically acceptable salt or isomer thereof. Also, it is another object to provide a use of a compound of formula (1), or a pharmaceutically acceptable salt or isomer thereof for cleansing or preserving a contact lens.

It is still another object of the invention to provide a composition for preserving an artificial intraocular lens, comprising a compound of formula (1), or a pharmaceutically acceptable salt or isomer thereof. Also, it is another object of the invention to provide a method of preserving an artificial intraocular lens, using a compound of formula (1), or a pharmaceutically acceptable salt or isomer thereof. Also, it is another object to provide a use of a compound of formula (1), or a pharmaceutically acceptable salt or isomer thereof for preserving an artificial intraocular lens.

### BRIEF DESCRIPTION OF THE DRAWINGS

FIG. 1 shows the comparison of damaged corneal areas via fluorescin staining in corneal wound models, wherein A shows the corneal area of a control group on the second day in which a physiological saline was applied to the corneal wound models; B shows the corneal area of a group which was treated with the inventive compound 1 on the second day; C shows the corneal area of the control group on the third day; and D shows the corneal area of the compound 1 treatment group on the third day. * shows a damaged area colored by a fluorescent dye. E, which quantifies the damaged areas of the corneas right after corneal wound induction and on the third day of the drug treatment, is a graph showing that the compound 1 treatment group exhibited a statistically significant (*** P<0.001) healing effect in comparison with the control group.

FIG. 2 shows the microscope observation results of corneal tissue fragments stained with Hematoxylin & Eosin in corneal wound models on the third day of drug treatment. A is a control group where a physiological saline was applied to the corneal wound model, and its epithelium in the portion marked with a red arrow remained damaged. B is a high-resolution picture of the square area of A, which shows clearly an area with restored epithelium and an unrestored area. C is a compound 1 treatment group of the corneal wound models, and its epithelium was completely restored. D is a high-resolution picture of the square area of C, which appears a normal corneal epithelium. A, C:×2.5; B, D:×400.

FIG. 3 shows electroretinography (ERG) responses in MNU (N-methyl-N-nitrosourea)-induced retinal degeneration models, in which A shows an a-wave, B shows a b-wave, and C shows a typical ERG response. The compound 1 treatment group shows a 200% higher ERG response than the control group.

FIG. 4 shows the microscope observation results of retinal tissue fragments stained with Hematoxylin & Eosin in MNU (N-methyl-N-nitrosourea)-induced retinal degeneration models on the fifth day of drug treatment. The control group showed the degeneration of photo receptor cells which are located at an outer nuclear layer (ONL) of the retina, the resultant destruction of the regularity of cell arrangement and decrease features in the number of cell layers as well as edema features in inner plexiform layer (IPL) in low-resolution (A) and high-resolution (C) pictures. The compound 1 treatment group showed a well-aligned retinal structure in low-resolution (B) and high-resolution (D) pictures. A, B: low resolution (×20). C, D: high resolution (×40).

FIG. 5 is the result which relatively shows the survived cells after analyzing through an MTT assay the apoptosis degrees obtained when a human retinal pigment epithelial cell line ARPE-19 was treated with NaIO₃ and then treated with compound 2 of the invention (+) or not treated therewith (−).

FIG. 6 (A) shows the result obtained when a white mouse retina was cultivated under explant culture conditions for 4 days, (B) shows the result obtained when a white mouse retina was cultivated under explant culture conditions for one day, then damaged with low oxygen in a hypoxic chamber for 30 min., and then cultivated for 3 days, and (C) shows the result obtained when a white mouse retina was cultivated under explant culture conditions for one day, then damaged with low oxygen in a hypoxic chamber for 30 min, treated with the compound 2 and then cultivated for 3 days. ONL represents an outer nuclear layer, INL represents an inner nuclear layer, and GCL represents a ganglion cell layer.

### DETAILED DESCRIPTION OF THE INVENTION

As one aspect for achieving such objects, the present invention relates to a pharmaceutical composition for preventing or treating an ophthalmologic disease comprising a compound of formula (1):

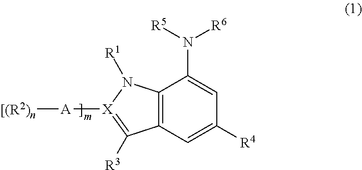

wherein

n is an integer of 1 to 3,

m is 0 or 1, with the proviso that when X is N, m is 0,

A represent phenyl,

X represents C or N,

R¹ represents hydrogen, C₁-C₆-alkyl or —(CH₂)ᵣNR⁷R⁸, wherein r is an integer of 2 to 5, and R⁷ and R⁸ are each independently hydrogen or C₁-C₃-alkyl, with the proviso that when X is N, R¹ is hydrogen,

R² represents hydrogen, halogen or a C₁-C₆-alkoxy group, represents —(CH₂)ₚCO₂R⁷, —(CH₂)ₚOR⁷, —(CH₂)ₚNR⁷R⁸, —NHR¹⁰, —N(H)S(O)₂R⁷ or —NHC(O)R¹⁰, or represents —(CH₂)ₚ-heterocycle-R¹⁰ which is a 5 to 6-membered ring in which the heterocycle portion contains 1 or 2 heteroatoms selected from N, O and S atoms, wherein p is an integer of 0 to 3, R⁷ and R⁸ are as defined above, and R¹⁰ represents hydrogen, oxo, C₁-C₆-alkylcarbonyl, C₁-C₆-alkoxy or C₁-C₆-alkyl or represents a 5 to 6-membered heterocycle containing 1 or 2 nitrogen atoms as a heteroatom,

R³ represents hydrogen, halogen, C₁-C₆-alkyl or phenyl, or represents —(CH₂)ₙ-heterocycle which a 5 to 6-membered ring containing 1 or 2 heteroatoms selected from N, O and S atoms wherein n is an integer of 0 to 3, with the proviso that when X is C and m is 0, R³ is phenyl, and when X is N, R³ is hydrogen or phenyl,

R⁴ represents —YR¹¹, wherein Y is a direct bond, or represents —(CR⁷R⁸)ₚY′—, wherein p is an integer of 0 to 3, R⁷ and R⁸ are as defined above,

Y′ is selected from the group consisting of —O—, —C(O)— and —C(O)O—, R¹¹ is selected from the group consisting of hydrogen, halogen, C₁-C₆-alkyl and —(CH₂)ₜB—R¹³, t is an integer of 0 to 3, B represents a 5 to 6-membered heterocycle containing 1 or 2 heteroatoms selected from N, 0 and S atoms, or represents C₆-C₁₀-aryl, and R¹³ represents hydrogen, cyano, halogen, hydroxy, oxo, thiol, carboxy or carboxy-C₁-C₆-alkyl, with the proviso that when X is N, R⁴ represents hydrogen or C₁-C₆-alkyl,

R⁵ represents hydrogen, C₁-C₆-alkyl, C₃-C₆-cycloalkyl, heterocycle or heterocyclyl-C₁-C₆-alkyl, wherein the heterocycle is a 3 to 8-membered ring containing 1 to 3 heteroatoms selected from N and O atoms, with the proviso that when X is N, R⁵ is hydrogen, and

R⁶ represents —(CR⁷R⁸)ₚ—Z-D-W—R¹⁴, wherein Z represents a direct bond, or is selected from the group consisting of —C(O)— and —C(O)O—, D represents a direct bond, represents C₄-C₆-cycloalkyl, represents a 5 to 6-membered heteroaryl containing 1 or 2 N atoms, or represents a 5 to 6-membered heterocycle containing 1 or 2 heteroatoms selected from N, 0 and S atoms, W represents a direct bond, or represents —NR⁷—, —C(O)—, —C(O)O—, —C(O)NR¹²— or —S(O)_{y}—, R¹² represents hydrogen, C₁-C₃-alkyl or C₆-C₁₀-aryl, y is an integer of 1 or 2, and R¹⁴ represents hydrogen, hydroxy, C₁-C₆-alkyl, a 5 to 6-membered heterocycle containing 1 or 3 heteroatoms selected from N, O and S atoms, or C₆-C₁₀-ar-C₁-C₆-alkyl,

with the proviso that when X is N, R⁶ represents C₄-C₆-cycloalkyl, or represents a 5 to 6-membered heterocycle containing 1 or 2 heteroatoms selected from N, O and S atoms,

wherein alkyl, alkoxy, aryl, cycloalkyl, heterocycle and heteroaryl may be optionally substituted, and the substituents are one or more selected from the group consisting of hydroxy, C₁-C₆-alkylamino, di(C₁-C₆-alkyl)amino, carboxy, C₁-C₆-alkyl, C₁-C₆-alkoxy, carboxy-C₁-C₆-alkyl and oxo,

or a pharmaceutically acceptable salt or optical isomer thereof.

In the above definitions for the compounds of formula (1) according to the invention, the term ‘alkyl’ means an aliphatic hydrocarbon radical. Alkyl may be saturated alkyl that does not comprise alkenyl or alkynyl moiety, or unsaturated alkyl that comprises at least one alkenyl or alkynyl moiety. “Alkenyl” means a group containing at least one carbon-carbon double bond, and “alkynyl” means a group containing at least one carbon-carbon triple bond. Alkyl may be branched or straight-chain when used alone or in a composite form such as alkoxy.

Alkyl group may have 1 to 20 carbon atoms unless otherwise defined. Alkyl group may be a medium sized alkyl having 1 to 10 carbon atoms. Otherwise, alkyl group may be a lower alkyl having 1 to 6 carbon atoms. Typical examples thereof include, but not limited to, methyl, ethyl, propyl, isopropyl, butyl, isobutyl, t-butyl, pentyl, hexyl, ethenyl, propenyl, butenyl, etc. For example, C₁-C₄-alkyl has 1 to 4 carbon atoms in the alkyl chain, and is selected from the group consisting of methyl, ethyl, propyl, iso-propyl, n-butyl, iso-butyl, sec-butyl and t-butyl.

The term ‘alkoxy’ means an alkyloxy having 1 to 10 carbon atoms unless otherwise defined. The term ‘cycloalkyl’ means a saturated aliphatic 3 to 10-membered ring unless otherwise defined. Typical examples thereof include, but not limited to, cyclopropyl, cyclobutyl, cyclopentyl, cyclohexyl, etc.

The term ‘aryl’ includes at least one ring having covalent pi electron system, for example, monocyclic or fused ring polycyclic groups (i.e., rings that share the adjacent carbon atom pairs). In the present specification, aryl means an aromatic 4 to 10-membered, preferably 6 to 10-membered, monocyclic or multicyclic ring including phenyl, naphthyl, etc., unless otherwise defined.

The term ‘heteroaryl’ means an aromatic 3 to 10-membered, preferably 4 to 8-membered, more preferably 5 to 6-membered ring that has 1 to 3 heteroatoms selected from N, O and S, and may be fused with benzo or C₃-C₈ cycloalkyl, unless otherwise defined. The monocyclic heteroaryl includes, but not limited to, thiazole, oxazole, thiophene, furan, pyrrole, imidazole, isoxazole, isothiazole, pyrazole, triazole, triazine, thiadiazole, tetrazole, oxadiazole, pyridine, pyridazine, pyrimidine, pyrazine and the like. The bicyclic heteroaryl includes, but not limited to, indole, indoline, benzothiophene, benzofuran, benzimidazole, benzoxazole, benzisoxazole, benzthiazole, benzthiadiazole, benztriazole, quinoline, isoquinoline, purine, puropyridine and the like.

The term ‘heterocycle’ means a 3 to 10-membered, preferably 4 to 8-membered, more preferably 5 to 6-membered ring that has 1 to 3 heteroatoms selected from N, O and S, may be fused with benzo or C₃-C₈ cycloalkyl, and is saturated or contains 1 or 2 double bonds, unless otherwise defined. The heterocycle includes, but not limited to, pyrroline, pyrrolidine, imidazoline, imidazolidine, pyrazoline, pyrazolidine, pyran, piperidine, morpholine, thiomorpholine, piperazine, hydrofuran and the like.

Other terms and abbreviations in the present specification may be understood to have the meaning conventionally used in this field by a skilled artisan, unless otherwise defined.

Preferred compounds among the compounds of formula (1) according to the invention are those wherein

n is an integer of 1 to 3,

m is 0 or 1, with the proviso that when X is N, m is 0,

A represents phenyl,

X represents C or N,

R¹ represents hydrogen, C₁-C₆-alkyl or —(CH₂)ᵣNR⁷R⁸, r is an integer of 2 to 3, R⁷ and R⁸ are each independently hydrogen or C₁-C₃-alkyl,

R² represents hydrogen, halogen, —(CH₂)ₚCO₂R⁷, —(CH₂)ₚOR⁷, —(CH₂)ₚNR⁷R⁸, —NHR¹⁰, —N(H)S(O)₂R⁷ or —NHC(O)R¹⁰, or represents —(CH₂)ₚ-heterocycle-R¹⁰ which is a 5 to 6-membered ring in which the heterocycle portion contains 1 or 2 heteroatoms selected from N and O atoms,

p is an integer of 0 to 3,

R¹⁰ represents hydrogen, oxo, C₁-C₆-alkylcarbonyl or C₁-C₆-alkyl, or represents a 5 to 6-membered heterocycle which contains 1 to 2 nitrogen atoms as a heteroatom and is optionally substituted by C₁-C₃-alkyl,

R³ represents hydrogen, halogen or C₁-C₆-alkyl, or represents phenyl optionally substituted by C₁-C₆-alkoxy, or represents heterocyclyl-C₁-C₃-alkylene which is a 5 to 6-membered ring wherein the heterocycle contains 1 to 2 heteroatoms selected from N and O atoms and is optionally substituted by 1 or 2 oxo groups, with the proviso that when X is C and m is 0, R³ is phenyl, and when X is N, R³ is hydrogen or phenyl,

R⁴ represent —YR¹¹, wherein Y is a direct bond or —(CR⁷R⁸)ₚY′—, Y′ is selected from the group consisting of —O—, —C(O)— and —C(O)O—, R¹¹ is selected from the group consisting of hydrogen, halogen, C₁-C₆-alkyl, hydroxy-C₁-C₆-alkyl and —(CH₂)ₜB—R¹³, t is an integer of 0 to 3, B represents C₆-C₁₀-aryl, or represents a 5 to 6-membered heterocycle containing 1 to 2 heteroatoms selected from N, O and S atoms, R¹³ represents hydrogen, halogen, hydroxy, oxo, thiol, carboxy or carboxy-C₁-C₆-alkyl,

R⁵ represents hydrogen, C₁-C₆-alkyl, C₃-C₆-cycloalkyl, heterocycle or heterocyclyl-C₁-C₆-alkyl, wherein the heterocycle is a 3 to 8-membered ring that contains 1 to 3 heteroatoms selected from N and O atoms and is optionally substituted by 1 or 2 oxo groups, with the proviso that when X is N, R⁵ is hydrogen,

R⁶ represents —(CR⁷R⁸)ₚ—Z-D-W—R¹⁴,

Z represents a direct bond, or is selected from the group consisting of —C(O)— and —C(O)O—,

D represents C₄-C₆-cycloalkyl, or represents a 5 to 6-membered heterocycle that contains 1 to 2 heteroatoms selected from N, O and S atoms and optionally contains an oxo group,

W represents a direct bond, or represents —NR⁷—. —C(O)—, —C(O)O—, —C(O)NR¹²— or —S(O)_{y}—, y is an integer of 1 or 2, R¹² represents hydrogen or C₁-C₃-alkyl, and

R¹⁴ represents hydrogen, hydroxy, C₁-C₆-alkyl, hydroxy-C₁-C₆-alkyl, carboxy-C₁-C₆-alkyl or C₆-C₁₀-ar-C₁-C₆-alkyl, or represents a 5 to 6-membered heterocycle that contains 1 to 3 heteroatoms selected from N, O and S atoms and is optionally substituted by 1 or 2 oxo groups, with the proviso that when X is N, R⁶ represents C₄-C₆-cycloalkyl, or represents a 5 to 6-membered heterocycle containing 1 to 2 heteroatoms selected from N, O and S atoms.

Preferably, X is C in the compounds of formula (1) according to the present invention, and the structure for this case may be depicted by the following formula (1a):

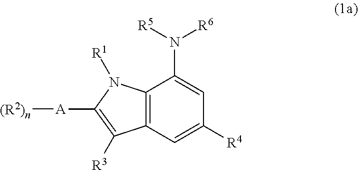

The substituent R¹ more preferably represents hydrogen, C₁-C₆-alkyl or di(C₁-C₃-alkyl)amino-C₂-C₃-alkyl, and most preferably represents hydrogen, methyl or (dimethylamino)ethyl.

The substituent R² more preferably represents hydrogen, amino, halogen, carboxy, carboxy-C₁-C₃-alkyl, C₁-C₃-alkoxycarbonyl, C₁-C₃-alkoxycarbonyl-C₁-C₃-alkyl, hydroxy-C₁-C₃-alkyl optionally substituted one oxo group, C₁-C₃-alkoxy, —(CH₂)ₚNR⁷R⁸, —NHR¹⁰, —N(H)S(O)₂R⁷ or —NHC(O)R¹⁰, or represents —(CH₂)ₚ-heterocycle-R¹⁰, wherein p, R⁷, R⁸ and R¹⁰ are as defined above and most preferably, it is selected from the group consisting of hydrogen, methoxy, fluoro, —NH₂, —NHAc, —NHSO₂Me, —NHBOC, —NH(1-methyl-piperidine), 1-oxo-2-hydroxy-ethyl, dimethylaminomethyl, hydroxymethyl, hydroxyethyl, carboxy, carboxymethyl, carboxyethyl, —CH₂-[(2-oxo)piperazine], —CH₂-piperazine, —CH₂-morpholine, —CH₂-[1,1-dioxo-thiomorpholine-4-yl] and —CH₂-[4-acetyl-piperazine-1-yl].

The substituent R³ more preferably represents hydrogen, methyl or bromo, represents phenyl optionally substituted by C₁-C₃-alkoxy, or represents heterocyclyl-C₁-C₃-alkylene which is a 5 to 6-membered ring that contains 1 or 2 heteroatoms selected from N and O atoms and is optionally substituted by 1 or 2 oxo groups, and most preferably, it is selected from the group consisting of hydrogen, methyl, bromo, phenyl, 4-MeO-phenyl, —CH₂-(2-oxo-piperazine-4-yl), and —CH₂-(morpholine-4-yl).

The substituent R⁴ more preferably represents —YR¹¹, wherein Y is selected from the group consisting of a direct bond, —O—, —C(O)—, —NH—, —CONH—, —SO₂NH—, —NHC(O)—, —CH₂CONH—, —CH₂C(O)—, and —CH₂SO₂—, and most preferably, Y is selected from the group consisting of a direct bond, —O—, —C(O)— and —CH₂C(O)—. Also, R¹¹ is selected from the group consisting of hydrogen, methyl, ethyl, phenyl, fluoro, chloro, 2-carboxy-pyrrolidine-1-yl, pyrrolidine-1-yl, 4-acetic acid-1,3-thiazoline-2-yl, —CH₂-(1,1-dioxo-thiomorpholine-4-yl) and —CH₂-(2-oxopiperazine-4-yl).

The substituent R⁵ more preferably represents hydrogen, C₁-C₆-alkyl, C₃-C₆-cycloalkyl, heterocycle or heterocyclyl-C₁-C₆-alkyl, wherein heterocycle represents a 5 to 6-membered ring that contains 1 or 2 heteroatoms selected from N and O atoms and is optionally substituted by 1 or 2 oxo groups, and most preferably, it is selected from the group consisting of hydrogen, methyl, cyclopentyl, tetrahydropyran-4-yl and CH₂-(tetrahydropyran-4-yl).

The substituent R⁶ represents —(CR⁷R⁸)ₚ—Z-D-W—R¹⁴, wherein Z represents a direct bond, or represents —C(O)—, —C(O)O— or —C(O)NH—. More preferably, D is selected from the group consisting of cyclopentyl, cyclohexyl, pyrrolidine, tetrahydropyran, tetrahydrofuran and piperidine. W represents a direct bond, or represents —SO₂—, —CO—, —C(O)O— or —CONR¹²— wherein R¹² is the same as defined in the above preferred examples. More preferably, the substituent W is selected from the group consisting of —SO₂—, —CO—, —C(O)O—, —CON(Me)- and —CONH—. R¹⁴ more preferably represents hydrogen, hydroxy, C₁-C₆-alkyl, hydroxy-C₁-C₆-alkyl or C₆-C₁₀-ar-C₁-C₃-alkyl, or represent a 5 to 6-membered heterocycle that contains one O or S atom and is optionally substituted by 1 or 2 oxo groups, and most preferably, it is selected from the group consisting of hydrogen, hydroxy, methyl, ethyl, isobutyl, hydroxymethyl, hydroxyethyl, tetrahydrofuran, tetrahydropyran and 1,1-dioxo-tetrahydro-thiopyran.

The compounds according to the invention can also form a pharmaceutically acceptable salt. Such a “pharmaceutically acceptable salt” includes a non-toxic acid addition salt containing a pharmaceutically acceptable anion, for example, a salt formed with inorganic acids such as sulfuric acid, hydrochloric acid, nitric acid, phosphoric acid, hydrobromic acid, hydriodic acid, etc.; a salt with organic carboxylic acids such as tartaric acid, formic acid, citric acid, acetic acid, trichloroacetic acid, trifluoroacetic acid, gluconic acid, benzoic acid, lactic acid, fumaric acid, maleic acid, salicylic acid, etc.; or a salt with sulfonic acids such as methanesulfonic acid, ethanesulfonic acid, benzenesulfonic acid, p-toluenesulfonic acid, naphthalenesulfonic acid, etc. Also a pharmaceutically acceptable base addition salt, for example, an alkali metal or alkaline earth metal salt formed from lithium, sodium, potassium, calcium, magnesium, etc.; an amino acid salt with lysine, arginine, guanidine, etc.; or an organic salt with dicyclohexylamine, N-methyl-D-glucamine, tris(hydroxymethyl)methylamine, diethanolamine, choline, triethylamine, etc. The compounds of formula (I) of the present invention may be converted to their salts according to any of the conventional methods, and the salt formation can be easily carried out by a skilled artisan based on the structure of formula (1) without additional explanations thereon.

The term ‘isomer’ in the present specification means those having the same chemical or molecular formula as, but optically or sterically different from, the compounds of formula (1), or salts thereof. The compounds of formula (1) of the present invention may have an asymmetric carbon center(s) in the structure, so that they may exist in the form of optical isomer (R or S isomer), racemate, mixture of diastereomers, or individual diastereomer, etc. When the compounds have a double bond, they may also exist in the form of geometric isomer (trans or cis isomer). All the isomers and their mixtures are also included in the scope of the present invention.

Hereinafter, the compounds of formula (1) include pharmaceutically acceptable salts and isomers thereof, unless otherwise explained. The salts and isomers should be construed to be included within the scope of the invention. For the sake of convenience, the present specification briefly expresses them as the compounds of formula (1).

Typical compounds of formula (1) according to the invention include the following compounds:
Cyclopentyl-[5-methyl-2-phenyl-1H-indole-7-yl]-amine;
4-[(5-Chloro-2-phenyl-1H-indole-7-yl)amino]-cyclohexane-1-one;
7-(Cyclopentyl)amino-2-phenyl-1H-indole-5-carboxylic acid ethyl ester;
Cyclopentyl-[5-hydroxymethyl-2-phenyl-1H-indole-7-yl]-amine;
7-(Cyclopentyl)amino-2-phenyl-1H-indole-5-carboxylic acid;
2-[7-(Cyclopentyl)amino-2-phenyl-1H-indole-5-yl]-acetic acid ethyl ester;
2-[7-(Cyclopentylamino)-2-phenyl-1H-indole-5-yl]ethanol;
2-[7-(Cyclopentyl)amino-2-phenyl-1H-indole-5-yl]acetic acid;
2-[2-Phenyl-7-(tetrahydropyran-4-yl)amino-1H-indole-5-yl]-acetic acid;
2-[2-Phenyl-7-(1,1-dioxo-tetrahydro-thiopyran-4-yl)amino-1H-indole-5-yl]-acetic acid;
(Tetrahydropyran-4-yl)-[2-phenyl-5-(1,1-dioxo-thiomorpholine-4-yl)methyl-1H-indole-7-yl]amine;
(Tetrahydropyran-4-yl)-[2-phenyl-5-(2-oxo-piperazine-4-yl)methyl-1H-indole-7-yl]amine;
Cyclopentyl-[2-(3-fluoro)phenyl-5-(2-oxo-piperazine-4-yl)methyl-1H-indole-7-yl]amine;
(Tetrahydropyran-4-yl)-[2-(4-methoxyl)phenyl-5-(1,1-dioxo-thiomorpholine-4-yl)methyl-1H-indole-7-yl]amine;
Cyclopentyl-[3,5-dimethyl-2-phenyl-1H-indole-7-yl]-amine;
(Tetrahydropyran-4-yl)-(5-methyl-2-phenyl-1H-indole-7-yl)-amine;
Cyclopentylmethyl-(5-methyl-2-phenyl-1H-indole-7-yl)-amine;
(Tetrahydropyran-4-ylmethyl)-(5-methyl-2-phenyl-1H-indole-7-yl)-amine;
(1-Methylpiperidine-4-yl)-(5-methyl-2-phenyl-1H-indole-7-yl)-amine;
1-[4-[(5-Methyl-2-phenyl-1H-indole-7-yl)amino]piperidine-1-yl]ethanone;
Cyclopentyl-(5-chloro-2-phenyl-1H-indole-7-yl)-amine;
Cyclohexyl-(5-chloro-2-phenyl-1H-indole-7-yl)-amine;
(Tetrahydropyran-4-yl)-(5-chloro-2-phenyl-1H-indole-7-yl)-amine;
Cyclopentylmethyl-(5-chloro-2-phenyl-1H-indole-7-yl)-amine;
(Tetrahydropyran-4-ylmethyl)-(5-chloro-2-phenyl-1H-indole-7-yl)-amine;
(1-Benzylpyrrolidine-3-yl)-(5-chloro-2-phenyl-1H-indole-7-yl)-amine;
(1-Methylpiperidine-4-yl)-(5-chloro-2-phenyl-1H-indole-7-yl)-amine;
(1,4-Dioxaspiro[4.5]decane-8-yl)-(5-chloro-2-phenyl-1H-indole-7-yl)-amine;
2-[(5-Chloro-2-phenyl-1H-indole-7-yl)amino]propane-1,3-diol;
(Tetrahydropyran-4-yl)-(5-methyl-2-phenyl-1H-indole-7-yl)-methyl-amine;
(Tetrahydropyran-4-ylmethyl)-[2-phenyl-5-(1,1-dioxo-thiomorpholine-4-yl)methyl-1H-indole-7-yl]amine;
Di(tetrahydropyran-4-ylmethyl)-[2-phenyl-5-(1,1-dioxo-thiomorpholine-4-yl)methyl-1H-indole-7-yl]amine;
Di(tetrahydropyran-4-yl)-[2-phenyl-5-(1,1-dioxo-thiomorpholine-4-yl)methyl-1H-indole-7-yl]amine;
(1-Methyl-piperidinemethyl-4-yl)-[5-fluoro-2-phenyl-1H-indole-7-yl]amine;
2-[4-[(5-Fluoro-2-phenyl-1H-indole-7-yl)amino]piperidine-1-yl]ethanol;
[1-(Tetrahydropyran-4-yl)piperidine-4-yl]-(5-fluoro-2-phenyl-1H-indole-7-yl)amine;
(Tetrahydropyran-4-yl)-(5-phenoxy-2-phenyl-1H-indole-7-yl)-amine;
(Tetrahydropyran-4-ylmethyl)-[2-phenyl-5-(2-oxo-piperazine-4-yl)methyl-1H-indole-7-yl]amine;
(Tetrahydropyran-4-yl)-[5-chloro-1-(2-diethylaminoethyl)-2-phenyl-1H-indole-7-yl]amine;
Dimethyl-(5-chloro-1-methyl-2-phenyl-1H-indole-7-yl)amine;
(Tetrahydropyran-4-yl)-(5-chloro-1-methyl-2-phenyl-1H-indole-7-yl)-methylamine;
(Tetrahydropyran-4-yl)-(5-chloro-3-phenyl-1H-indole-7-yl)-amine;
Cyclopentyl-(5-chloro-3-phenyl-1H-indole-7-yl)-amine;
(Tetrahydropyran-4-ylmethyl)-(5-chloro-3-phenyl-1H-indole-7-yl)-amine;
Cyclopentyl-(5-chloro-3-(morpholine-4-yl)methyl-2-phenyl-1H-indole-7-yl)-amine;
(Tetrahydropyran-4-yl)-(5-chloro-3-(morpholine-4-yl)methyl-2-phenyl-1H-indole-7-yl)-amine;
(Tetrahydropyran-4-yl)-(5-chloro-3-(2-oxo-piperazine-4-yl)methyl-2-phenyl-1H-indole-7-yl)-amine;
Cyclopentyl-(5-chloro-3-(2-oxo-piperazine-4-yl)methyl-2-phenyl-1H-indole-7-yl)-amine;
(Tetrahydropyran-4-ylmethyl)-(5-chloro-3-(2-oxo-piperazine-4-yl)methyl-2-phenyl-1H-indole-7-yl)-amine;
(Tetrahydropyran-4-yl)-(3-bromo-5-(1,1-dioxo-thiomorpholine-4-yl)methyl-2-phenyl-1H-indole-7-yl)-amine;
Cyclopentyl-(3-bromo-5-(1,1-dioxo-thiomorpholine-4-yl)methyl-2-phenyl-1H-indole-7-yl)-amine;
(Tetrahydropyran-4-ylmethyl)-(3-bromo-5-(1,1-dioxo-thiomorpholine-4-yl)methyl-2-phenyl-1H-indole-7-yl)-amine;
(Tetrahydropyran-4-yl)-(5-(1,1-dioxo-thiomorpholine-4-yl)methyl-2-(3-fluorophenyl)-1H-indole-7-yl)-amine;
(Tetrahydropyran-4-yl)-(5-chloro-3-phenyl-1H-indole-7-yl)-amine;
(3-Methylbutyl)-[5-(1,1-dioxo-thiomorpholine-4-yl)methyl-2-(4-methoxyphenyl)-1H-indole-7-yl]-amine;
t-Butyl N-[4-[5-chloro-7-(cyclopentylamino)-1H-indole-2-yl]phenyl]carbamate;
Cyclopentyl-[2-(4-aminophenyl)-5-chloro-1H-indole-7-yl]-amine;
Cyclopentyl-{5-chloro-2-[4-(1-methyl-piperidine-4-yl)aminophenyl]-1H-indole-7-yl}-amine;
N-[4-(5-Chloro-7-cyclopentylamino-1H-indole-2-yl)-phenyl]-methanesulfoneamide;
Cyclopentyl-{5-(1,1-dioxo-thiomorpholine-4-yl)methyl-2-[4-(acetyl)aminophenyl]-1H-indole-7-yl}-amine;
Dicyclopentyl-{5-(1,1-dioxo-thiomorpholine-4-yl)methyl-2-[4-(acetyl)aminophenyl]-1H-indole-7-yl}-amine;
(Tetrahydropyran-4-yl)methyl-{5-(1,1-dioxo-thiomorpholine-4-yl)methyl-2-[4-(acetyl)aminophenyl]-1H-indole-7-yl}-amine;
Di(tetrahydropyran-4-yl)methyl-{5-(1,1-dioxo-thiomorpholine-4-yl)methyl-2-[4-(acetyl)aminophenyl]-1H-indole-7-yl}-amine;
(Tetrahydropyran-4-yl)-{5-(1,1-dioxo-thiomorpholine-4-yl)methyl-2-[4-(acetyl)aminophenyl]-1H-indole-7-yl}-amine;
(5-Methyl-2-phenyl-1H-indole-7-yl)-piperidine-4-yl-amine;
[1-(Methanesulfonyl)piperidine-4-yl]-(5-methyl-2-phenyl-1H-indole-7-yl)-amine;
2-Hydroxy-1-[4-(5-methyl-2-phenyl-1H-indole-7-yl)amino-piperidine-1-yl]-ethanone;
(5-Chloro-2-phenyl-1H-indole-7-yl)-piperidine-4-yl-amine;
4-(5-Chloro-2-phenyl-1H-indole-7-yl)amino-piperidine-1-yl-carboxylic acid phenylamide;
1-[4-(5-Chloro-2-phenyl-1H-indole-7-yl)amino-piperidine-1-yl]-2-dimethylamino-ethanone;
[5-(1,1-Dioxo-thiomorpholine-4-yl)methyl-2-phenyl-1H-indole-7-yl]-(piperidine-4-yl)methyl-amine;
(5-(1,1-Dioxo-thiomorpholine-4-yl)methyl-2-phenyl-1H-indole-7-yl)-(1-methanesulfonyl-piperidine-4-yl)-amine;
{4-[5-(1,1-Dioxo-thiomorpholine-4-yl)methyl-2-phenyl-1H-indole-7-yl]amino-piperidine-1-yl}-(tetrahydrofuran-2-yl)-methanone;
(5-Fluoro-2-phenyl-1H-indole-7-yl)-[1-(1,1-dioxo-tetrahydrothiopyran-4-yl)-piperidine-4-yl]-amine;
N-(5-Chloro-2-phenyl-1H-indole-7-yl)-N,N-dimethyl-cyclohexane-1,4- [symbol] amine;
N-(5-Chloro-2-phenyl-1H-indole-7-yl)-N′-methyl-cyclohexane-1,4- [symbol] amine;
4-(5-Chloro-2-phenyl-1H-indole-7-yl)amino-cyclohexane-1-carboxylic acid;
4-(5-Methyl-2-phenyl-1H-indole-7-yl)amino-cyclohexane-1-carboxylic acid;
4-(5-Chloro-2-phenyl-1H-indole-7-yl)amino-cyclohexane-1-carboxylic acid amide;
4-(5-Chloro-2-phenyl-1H-indole-7-yl)amino-cyclohexanecarboxylic acid methylamide;
2-(5-Fluoro-2-phenyl-1H-indole-7-yl)amino-acetic acid methyl ester;
2-(5-Fluoro-2-phenyl-1H-indole-7-yl)amino-acetic acid;
2-(5-Phenoxy-2-phenyl-1H-indole-7-yl)amino-acetic acid methyl ester;
2-[(5-Phenoxy-2-phenyl-1H-indole-7-yl)amino]-acetic acid;
2-[(5-Phenoxy-2-phenyl-1H-indole-7-yl)amino]-propionic acid methyl ester;
2-(5-Phenoxy-2-phenyl-1H-indole-7-yl)amino-propionic acid;
2-(5-Chloro-2-phenyl-1H-indole-7-yl)amino-propionic acid;
(5-Chloro-2-phenyl-1H-indole-7-yl)-pyridine-2-yl-amine;
(5-Chloro-2-phenyl-1H-indole-7-yl)-5-methyl-pyridine-2-yl-amine;
(5-Chloro-3-phenyl-1H-indole-7-yl)-(5-methyl-pyridine-2-yl)-amine;
(2S)-1-(7-Cyclopentylamino-2-phenyl-1H-indole-5-carbonyl)-pyrrolidine-2-carboxylic acid methyl ester;
(2S)-1-(7-Cyclopentylamino-2-phenyl-1H-indole-5-carbonyl)-pyrrolidine-2-carboxylic acid;
(2S)-1-[2-Phenyl-7-(tetrahydropyran-4-yl)amino-1H-indole-5-carbonyl]-pyrrolidine-2-carboxylic acid;
2-(7-Cyclopentylamino-2-phenyl-1H-indole-5-yl]-1-pyrrolidine-1-yl-ethanone;
Cyclopentyl-[2-phenyl-5-(2-pyrrolidine-1-yl-ethyl)-1H-indole-7-yl]-amine;
2-[(R)-2-(7-Cyclopentylamino-2-phenyl-1H-indole-5-yl)-4,5-dihydro-thiazole-4-yl]-acetic acid;
2-[(R)-2-(2-Phenyl-7-(tetrahydropyran-4-yl)methylamino-1H-indole-5-yl)-4,5-dihydro-thiazole-4-yl]acetic acid;
3-(7-Cyclopentylamino-5-chloro-1H-indole-2-yl)-benzoic acid methyl ester;
3-(7-Cyclopentylamino-5-chloro-1H-indole-2-yl)-benzoic acid;
[3-(5-Chloro-7-cyclopentylamino-1H-indole-2-yl)-phenyl]-methanol;
{3-[5-Chloro-7-(tetrahydropyran-4-yl)amino-1H-indole-2-yl]-phenyl}-methanol;
2-{3-[5-Chloro-7-(tetrahydropyran-4-ylmethyl)amino-1H-indole-2-yl]-phenyl}-acetic acid;
2-[3-(5-Chloro-7-cyclopentylamino-1-H-indole-2-yl)-phenyl]-acetic acid;
2-{3-[5-Chloro-7-(tetrahydropyran-4-yl)amino-1H-indole-2-yl]-phenyl}-acetic acid;
2-{3-[5-Chloro-7-(tetrahydropyran-4-ylmethyl)amino-1-H-indole-2-yl]-phenyl}-acetic acid methyl ester;
2-[3-(5-Chloro-7-cyclopentylamino-1H-indole-2-yl)-phenyl]-acetic acid methyl ester;
2-{3-[5-Chloro-7-(tetrahydropyran-4-yl)amino)-1H-indole-2-yl]-phenyl}-acetic acid methyl ester;
[2-(5-Chloro-7-cyclopentylamino-1H-indole-2-yl)-phenyl]-methanol;
2-(5-Chloro-7-cyclopentylamino-1H-indole-2-yl)-benzoic acid;
2-(5-Chloro-7-cyclopentylamino-1H-indole-2-yl)-benzoic acid methyl ester;
[4-(5-Chloro-7-cyclopentylamino-1H-indole-2-yl)-phenyl]-methanol;
2-{4-[5-Chloro-7-(tetrahydropyran-4-yl)amino-1H-indole-2-yl]phenyl}-ethanol;
4-(5-Chloro-7-cyclopentylamino-1H-indole-2-yl)-benzoic acid;
2-{4-[5-Chloro-7-(tetrahydropyran-4-ylmethyl)amino-1H-indole-2-yl]phenyl}-acetic acid;
2-[4-(5-Chloro-7-cyclopentylamino-1H-indole-2-yl)phenyl]-acetic acid;
2-{4-[5-Chloro-7-(tetrahydropyran-4-yl)amino-1H-indole-2-yl]phenyl}-acetic acid;
4-(5-Chloro-7-cyclopentylamino-1H-indole-2-yl)benzoic acid methyl ester;
4-(7-Cyclopentylamino-5-methyl-1H-indole-2-yl)benzoic acid methyl ester;
2-{4-[5-Chloro-7-(tetrahydropyran-4-ylmethyl)amino-1H-indole-2-yl]phenyl}-acetic acid methyl ester;
2-[4-(5-Chloro-7-cyclopentylamino-1H-indole-2-yl)phenyl]acetic acid methyl ester;
2-{4-[5-Chloro-7-(tetrahydropyran-4-yl)amino-1H-indole-2-yl]phenyl}-acetic acid methyl ester;
[5-Chloro-2-(3-dimethylaminomethylphenyl)-1H-indole-7-yl]-(tetrahydropyran-4-yl)-amine;
[5-Chloro-2-(3-morpholine-4-ylmethylphenyl)-1H-indole-7-yl]-(tetrahydropyran-4-yl)-amine;
1-{4-[3-(5-Chloro-7-tetrahydropyran-4-ylamino-1H-indole-2-yl)-benzyl]-piperazine-1-yl}-ethanone;
{5-Chloro-2-[3-(2-oxo-piperazine-4-yl)ethylphenyl]-1H-indole-7-yl}-(tetrahydropyran-4-yl)-amine;
{5-Chloro-2-[3-(1,1-dioxo-thiomorpholine-4-yl)ethylphenyl]-1H-indole-7-yl}-(tetrahydropyran-4-yl)-amine;
Cyclopentyl-(1H-indazole-7-yl)-amine;
(Tetrahydropyran-4-yl)-(1H-indazole-7-yl)-amine;
Cyclopentyl-(5-methyl-1H-indazole-7-yl)-amine;
(5-Methyl-1H-indazole-7-yl)-(tetrahydropyran-4-yl)-amine;
Cyclopentyl-[3-(4-methoxyphenyl)-1H-indazole-7-yl)-amine;
[3-(4-Methoxyphenyl)-1H-indazole-7-yl)]-(tetrahydropyran-4-yl)-amine;
[3-(4-Methoxyphenyl)-1H-indazole-7-yl)-(tetrahydropyran-4-ylmethyl)-amine;
(Tetrahydropyran-4-yl)-[5-(1,1-dioxo-thiomorpholine-4-yl)methyl-3-phenyl-2-trimethylsilyl-1H-indole-7-yl)-amine;
(Tetrahydropyran-4-yl)-[5-(1,1-dioxo-thiomorpholine-4-yl)methyl-3-phenyl-1H-indole-7-yl)-amine; and
(Tetrahydropyran-4-yl)-[3-bromo-5-(morpholine-4-yl)methyl-2-phenyl-1H-indole-7-yl]-amine.

More preferably, the compound of formula (1) according to the invention may be depicted by the following formula (1b):

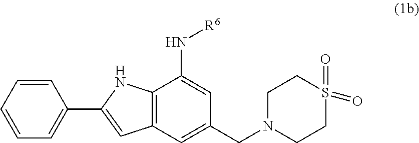

wherein

R⁶ represents —(CR⁷R⁸)ₚ—Z-D-W—R¹⁴,

R⁷ and R⁸ each independently represent hydrogen or C₁-C₃-alkyl, wherein p is an integer of 0 or 1,

Z represents a direct bond,

D represents a 5 to 6-membered heterocycle containing N or O atom and more preferably, it is tetrahydropyran or piperidine,

W represents a direct bond, or represents —S(O)_{y}—, wherein y is an integer of 1 or 2, and

R¹⁴ represents hydrogen or C₁-C₆-alkyl.

More preferably, the compounds according to the above formula (1b) may include the following compounds:
(Tetrahydropyran-4-yl)-[2-phenyl-5-(1,1-dioxo-thiomorpholine-4-yl)methyl-1H-indole-7-yl]amine;
(Tetrahydropyran-4-ylmethyl)-[2-phenyl-5-(1,1-dioxo-thiomorpholine-4-yl)methyl-1H-indole-7-yl]amine; and
(5-(1,1-Dioxo-thiomorpholine-4-yl)methyl-2-phenyl-1H-indole-7-yl)-(1-methanesulfonyl-piperidine-4-yl)-amine.

For methods of preparing the above compounds, the methods disclosed in Korea patent laid-open No. 10-2009-0018593 may be referred to and the entire content of the above patent document is incorporated into this specification by reference.

The present invention is characterized by providing a use of a composition comprising a compound of formula (1), or a pharmaceutically acceptable salt or isomer thereof for preventing and treating an ophthalmologic disease.

As used herein, “treatment” means the interrupting or delaying the progress of a disease when applied to the subject showing the symptoms, and “prevention” means the interrupting or delaying the sign of the onset of the disease when applied to the subject that does not show, but is at high risk of the onset of disease symptoms.

The ophthalmologic diseases to which the compositions of the present invention are applicable may include all the diseases related to eyeballs. Preferably, the ophthalmologic diseases in the invention include, as retina-related diseases, acute and chronic degenerative diseases of retina-related cells or tissues. Preferred examples thereof include glaucoma, retinal degeneration, retinal pigment degeneration, retinal detachment, retinal tear, retinal ischemic disease and diabetic retinopathy.

Also, the ophthalmologic diseases in the invention include cataract.

Further, the ophthalmologic diseases in the invention include, as a cornea-related disease, keratoconjunctival epithelial damage or corneal epithelial wound. The keratoconjunctival epithelial damage means a damage of corneal epithelial cells which constitute a corneal epithelial layer at the very surface layer of the cornea, and includes keratoconjunctival epithelial damages due to endogenous diseases such as corneal herpes, corneal ulcer, keratitis, conjunctivitis, diabetic keratopathy, Sjogren's syndrome, xerophthalmia, etc., and keratoconjunctival epithelial damages due to exogenous diseases such as alcohol, drugs, injury, contact lens wear, etc. The corneal epithelial wound refers to a wound in a broad sense including all kinds of injuries resulted from the tear, incision, or perforations of corneal epithelial tissues. Examples of the corneal epithelial wound include, but not limited to, a wound due to xerotic keratitis (xerophthalmia) caused by the dryness of a corneal surface due to the reduction of tear secretion or an unstable tear film; a wound due to infectious keratitis by bacterial or viral infection; a wound due to the eyeball invasion of a systemic disease; a secondary wound caused by chronic conjunctivitis (allergy conjunctivitis), corneal ulcer, etc.; a wound due to corneal surgery for example laser refractive surgery for vision correction such as Lasek surgery or Epi-LASIK surgery; and a wound after corneal transplantation.

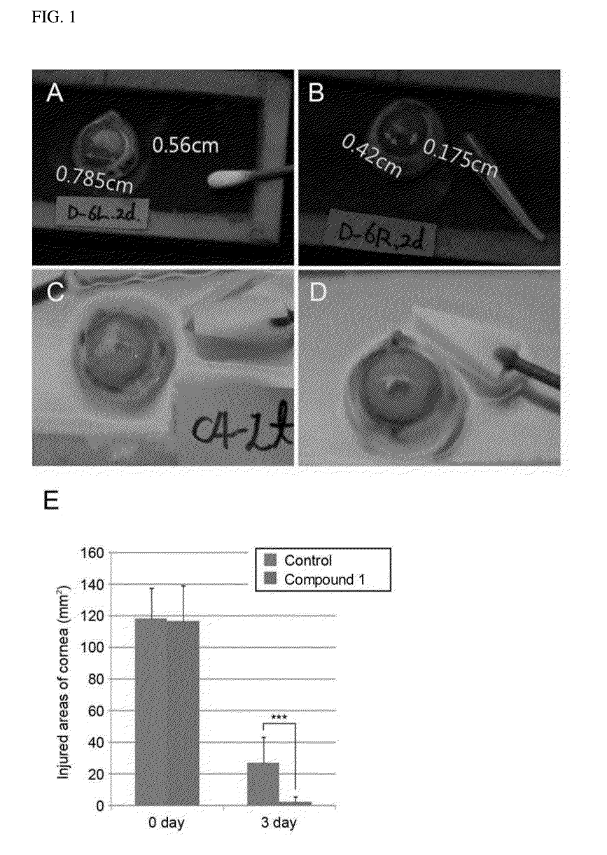

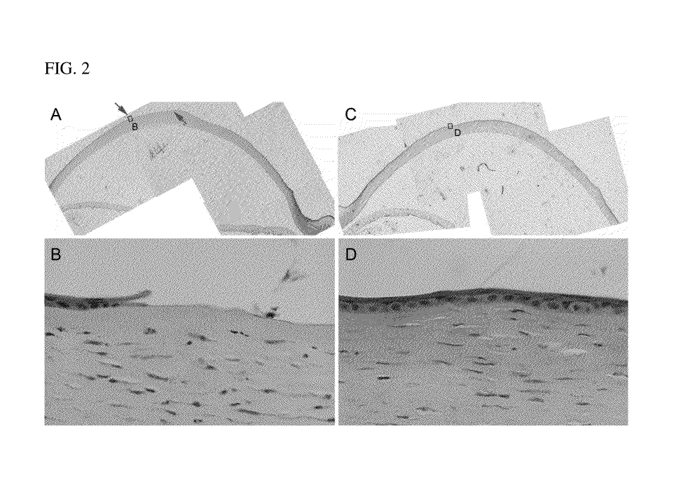

In the specific examples of the present invention, to see whether the compounds of formula (1) show stable corneal wound healing effects in ophthalmologic operations such as LRP (laser reversal of presbyopia), LASIK (laser assisted in-situ keratomileusis) and LASEK (laser assisted sub-epithelial keratomileusis), a corneal wound animal model in which corneal epithelium was removed using a rotary epithelial scrubber was prepared and then, the composition of the invention and a physiological saline as a control group were applied thereto, respectively to measure corneal wound healing effects. Corneal wound areas were examined using fluorescin staining and as a result, the group to which the composition of the invention was applied showed a quicker healing effect than the control group, and the microscope observation of the corneal tissue fragments also confirmed that the corneal epithelium was completely restored on the 3^{rd} day of application (FIG. 1 and FIG. 2).

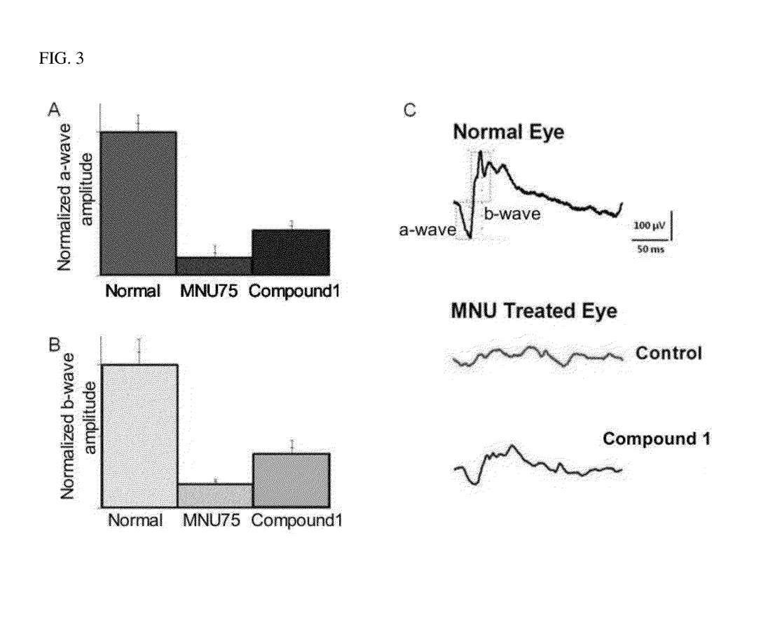

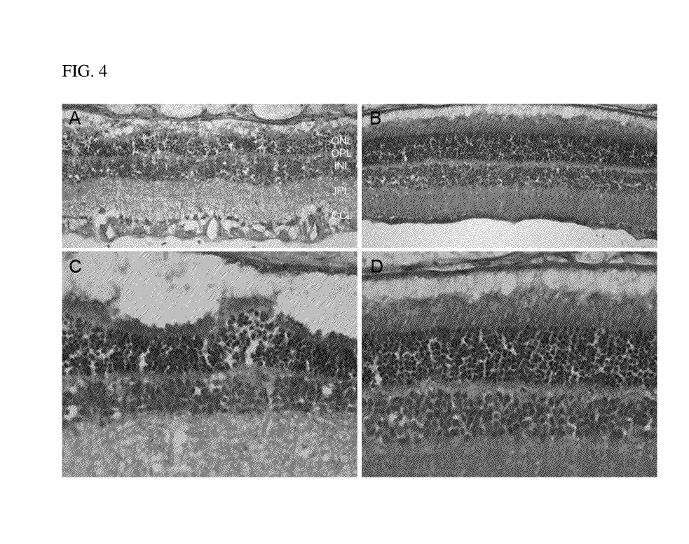

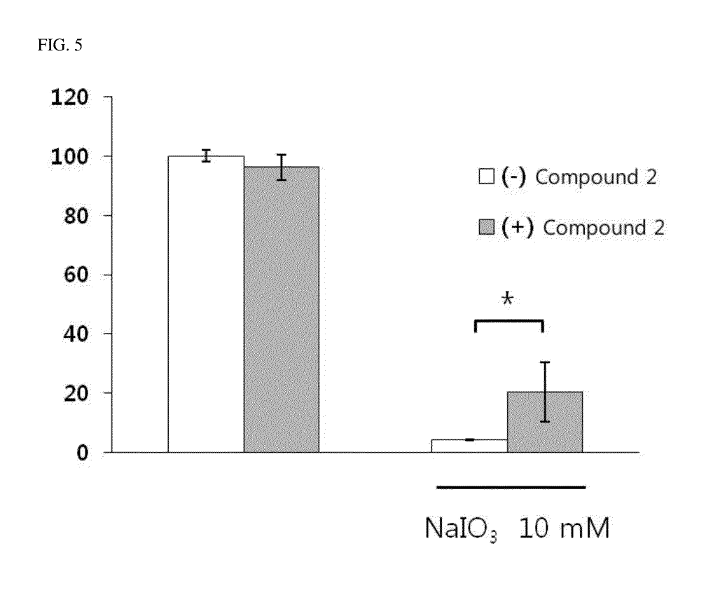

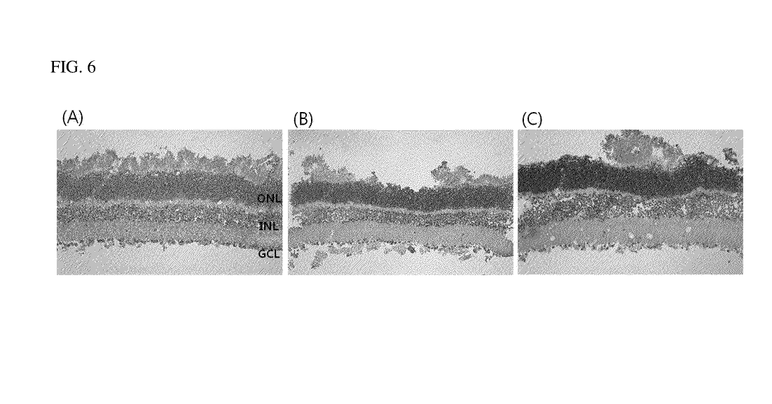

Further, to evaluate the efficiency of the compounds of formula (1) in retinal degeneration which is hard to cure or incurable, a retinal degeneration animal model in which retinal degeneration was induced via the intraperitoneal injection of MNU (N-methyl-N-nitrosourea) was prepared and then, the composition of the invention and a physiological saline as a control group were applied thereto, respectively to measure retina healing effects. As a result of the evaluation of electroretinogram, the group to which the composition of the invention was applied showed an increased healing effect in comparison with the control group, and the microscope observation of the retinal tissue fragments also confirmed that they had very close normal tissue features on the 5^{th} day of application (FIG. 3 and FIG. 4). In addition, it was confirmed that retinal cells also showed apoptosis suppression effects and the healing effects of cell damage resultant from a low oxygen condition (FIG. 5 and FIG. 6).

Accordingly, the compositions of the invention can be usefully applied to prevent and treat ophthalmologic diseases.

The above-mentioned “pharmaceutical composition” may comprise a pharmaceutically acceptable carrier, diluent, excipient, or a combination thereof, if needed, together with the compounds of the present invention. A pharmaceutical composition facilitates the administration of a compound into a living organism. There exist a number of techniques to administer a compound, and they include, but not limited to, oral, injectable, aerosol, parenteral and topical administration.

The compositions of the invention may further comprise a pharmaceutically acceptable carrier or additive. The pharmaceutically acceptable carrier means a carrier or diluent which considerably does not stimulate a living organism and does not inhibit the biological activities and properties of the compound to be administered. Also, the additive may facilitate the preparation, compressibility, appearance and flavor of the formulation and for example, a stabilizer, a surfactant, a slip modifier, a solubilizing agent, a buffering agent, a sweetening agent, a base compound, an absorbent, a flavor enhancer, a binding agent, a suspending agent, a hardening agent, an anti-oxidant, a polishing agent, a fragrance ingredient, a flavoring agent, a pigment, a coating agent, a wetting agent, a moisture adjusting agent, a filler, an antifoaming agent, a refreshing agent, a masticating agent, an antistatic agent, a coloring agent, a sugar coating agent, an isotonic agent, a softening agent, an emulsifying agent, a sticking agent, a thickening agent, a foaming agent, a pH adjusting agent, an excipient, a dispersing agent, a disintegrating agent, a waterproof agent, an antiseptic agent, a preservative, a solubilizing aid, a solvent, a plasticizer, etc. may be added, if needed.

The dosage of the compounds of formula (1) depends on the prescription of a physician, taking into account such factors as body weight, sex, age, condition of health, and diet of the patient, specific nature of the disease, administration time of the drug, administration method, mixing ratio of drugs, and severity of the disease, etc. However, dosage needed for the treatment of an adult is typically from about 1.0 mg to 2,000 mg per day, depending on the intensity and frequency of the administration. When administered to an adult via intramuscular or intravenous routes, total dosage typically from about 1.0 mg to 300 mg per day will be sufficient when separately administered in a single dosage, but for some patients a higher daily dosage may be desirable.

As another aspect, the invention relates to a composition for cleansing or preserving a contact lens comprising a compound of formula (1), or a pharmaceutically acceptable salt or isomer thereof.

In the case of the composition for cleansing a contact lens, a surfactant may be included as a major ingredient and the compound represented by the above formula (1), or a salt or isomer thereof may be included as a supplemental ingredient. The surfactants having cleansing effects may comprise various surfactants known in the pertinent art, including anionic, cationic, non-ionic and amphiprotic surfactants, as a major cleansing agent. Also, a wetting agent, an antimicrobial agent, a stabilizer, an isotonic agent, a solubilizing aid, a viscosity adjuster or a buffering solution may be additionally included.

In the case of the composition for preserving a contact lens, salt water, other buffering solutions or deionized water may be included as an aqueous solution for storing the contact lens, besides the compound represented by formula (1) and a salt or isomer thereof, and preferably, a boron-based buffering agent such as boric acid, borax etc., an acetate-based buffering agent such as acetic acid, sodium acetate, potassium acetate, etc., a phosphate-based buffering agent such as sodium hydrogen phosphate, sodium dihydrogen phosphate, potassium dihydrogen phosphate, etc., a carbonate-based buffering agent such as sodium carbonate, sodium hydrogen carbonate, etc, a citrate-based buffering agent such as citric acid, sodium citrate, etc. or tromethanmol buffering agent may be included. More preferably, salt-containing salt water including sodium chloride, sodium borate, sodium phosphate, sodium hydrogen phosphate, sodium dihydrogen phosphate, or their corresponding potassium salts may be included. Further, a wetting agent, a surfactant, a stabilizer, a viscosity adjuster, an isotonic agent, a solubilizing aid, an antioxidant, an antiseptic agent, a refreshing agent, a chelating agent, a tenderizing agent and so on may be additionally included.

As another aspect, the invention relates to a composition for preserving an artificial intraocular lens, comprising a compound of formula (1), or a pharmaceutically acceptable salt or isomer thereof.

The artificial intraocular lens is a lens used to replace the original lens when the lens of a patient is infected with a disease or it is damaged, and mostly it is implanted to the eye to replace the original lens which is removed from the eye during cataract surgery. Since the artificial intraocular lens is to be used in a human body, it is essential to safely preserve it from being infected or contaminated till the implantation. Since the composition of the invention shows healing effects related to ophthalmologic diseases or eyeball damages, it can be included in a preserving solution for an artificial intraocular lens so that it can protect the artificial intraocular lens from external infection or contamination and it can function not to cause endophthalmitis during implantation into the human body. The composition for preserving an artificial intraocular lens of the invention may further comprise a wetting agent, an antimicrobial agent, a stabilizer, an isotonic agent, a solubilizing aid, a viscosity adjuster, an antioxidant or a buffering solution.

The invention will be described in more detail by examples. The following examples are intended to merely illustrate the present invention, and the scope of the invention is not limited by them in any ways.

### Example 1

### Corneal Wound Healing Effect Test

It was examined whether the compounds of formula (1) show clinically stable corneal wound healing effects in ophthalmologic surgery such as LRP (laser reversal of presbyopia), LASIK (laser assisted in-situ keratomileusis) and LASEK (laser assisted sub-epithelial keratomileusis). For this, 16 New Zealand white rabbits of 2.0˜2.5 kg were prepared as test animals, anesthetized via intramuscular injection with 15 mg/kg of Zoletil and 5 mg/kg of Xylazine, and then their corneal epitheliums with diameters of 10-12 mm or so were removed using a surgery blade (No. 15) and a rotary epithelial scrubber to produce corneal wound models. At 30 min. after the above-mentioned surgery, two drops of compound 1, that is, (tetrahydropyran-4-ylmethyl)-[2-phenyl-5-(1,1-dioxo-thiomorpholine-4-yl)methyl-1H-indole-7-yl]amine) (KDR Biotech. Co., Ltd., Seoul, Korea) of 50 μM were applied to the experimental group and two drops of a physiological saline were applied to the control group and then, they wore contact lenses, and one drop of the same medicines was applied at 12-hour intervals. To measure a wound area in the experimental group and the control group, their corneal surfaces were stained with Fluorescin using Fluorescin (Haag-Streit International, Switzerland) strips and their diameters were measured using a microcaliper at 24-hour intervals. In the meantime, for the histological evaluation of the experimental group and the control group, they were anesthetized with 25% urethane on the first, second and third day to extract their eyeballs and then, their anterior eye parts including cornea were embedded in wax (Polyscience, USA) to produce 5-μm fragments, which were then stained with Hematoxylin & Eosin and observed using a microscope.

As a result of Fluorescin stain, the control group to which the physiological saline was applied after the preparation of the corneal wound model was healed on the 4^{th} to 5^{th} day whereas the experimental group to which the compound 1 was applied was healed within the 3 days (FIG. 1) and also, in the microscope observation of the corneal tissue fragments, epitheliums still remained damaged in the control groups on the 3^{rd} day after the preparation of the corneal wound model whereas in the compound 1 treatment group, the epitheliums were completely restored, confirming the healing effect of compound 1 (FIG. 2).

### Example 2

### Retinal Nerve Protection Effect Test

2-1. Efficiency Test in Retinal Degeneration Animal Model

The compounds of formula (1) were applied to retinal degeneration which is hard to cure or incurable to evaluate their effects. For this, 3 250-g Sprague-Dawley rats were prepared as a test animal, and injected with 75 mg/kg of N-methyl-N-nitrosourea (MNU) via an intraperitoneal route to induce retinal degeneration, thereby to produce retinal degeneration models. At 30 min. after the retinal degeneration induction, 10 μl of compound 1, that is, (tetrahydropyran-4-ylmethyl)-[2-phenyl-5-(1,1-dioxo-thiomorpholine-4-yl)methyl-1H-indole-7-yl]amine) (KDR Biotech. Co., Ltd., Seoul, Korea) of 50 μM and a physiological saline was injected into the vitreous body of the experimental group and the control group, respectively and then, the same medicines were injected via the same methods after 72 hours of the retinal degeneration induction. To evaluate the healing effects of retinal degeneration in the experimental group and the control group, on the 5^{th} day of the retinal degeneration induction, the animals were anesthetized using 8% chloral hydrate (0.5 mg/kg) and subjected to electroretinogram (ERG) test (UTAS-2000; LKC Technologies, USA), in which scotopic response was recorded as a single flash response having a strength of 0.9 log (cd s) m⁻² or higher. In the meantime, for the histological evaluation of the experimental group and the control group, their eyeballs were extracted from the animals which completed the electroretinogram test on the 5^{th} day of the retinal degeneration induction to produce eyecups, and then they were embedded in wax to produce 5-μm fragments, which were then stained with Hematoxylin & Eosin and observed using a microscope.

As a result of the electroretinogram test, the compound 1 treatment group showed an increased response in both a- and b-waves compared to the control group (FIG. 3), and in the microscope observation results of the retinal tissue fragments, the control group showed a severe retinal degeneration feature whereas the compound 1 treatment group appeared close to a normal tissue (FIG. 4).

2-2. Protection Efficiency Test of Retinal Pigment Epithelial Cell

A human retinal pigment epithelial cell line ARPE-19 was treated with 10 mM of NaIO₃, which is a retinal pigment epithelial cell apoptosis inducing agent, to induce apoptosis and then, it was treated with compound 2 of the invention, that is, (5-(1,1-dioxo-thiomorpholine-4-yl)methyl-2-phenyl-1H-indole-7-yl)-(1-methanesulfonyl-piperidine-4-yl)-amine (Enzo Life Sciences, Inc.) of 1 μM, and the compound treatment group and the non-treatment group were analyzed about their apoptosis degree using an MTT assay and survived cells were comparatively analyzed. The results are shown in FIG. 5, and the compound 2 treatment group showed statistically significant (* P<0.05; Student's t-test) survival results in comparison with the control group.

2-3. Retinal Nerve Cell Protection Efficiency Test on Low Oxygen Stimulation

A white rat retina was cultivated overnight under explant culture conditions and then, damaged with low oxygen in a hypoxic chamber for 30 min., and cultivated for 3 days with or without being treated with compound 2 of 1 μM, and after the cultivation, the obtained retinal explants were stained and observed using a microscope. The results are shown in FIG. 6, and the retinal explant damaged with low oxygen in the hypoxic chamber showed thinner outer nuclear layer (ONL) and inner nuclear layer (INL) than the non-damaged explant, whereas in the compound 2 treatment group which was damaged with the same low oxygen but was treated with 1 μM compound 2, it was well preserved.

The compositions of the invention can be usefully applied to prevent and treat ophthalmologic diseases and they are also applicable to compositions for cleansing or preserving a contact lens and compositions for preserving an artificial intraocular lens.

1. A method for treating an ophthalmologic disease, comprising administering to a subject a therapeutically effective amount of an indole or indazole compound of formula (1), or a pharmaceutically acceptable salt or isomer thereof: 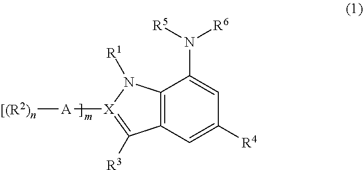
wherein
n is an integer of 1 to 3,
m is 0 or 1, with the proviso that when X is N, m is 0,
A represent phenyl,
X represents C or N,
R¹ represents hydrogen, C₁-C₆-alkyl or —(CH₂)ₚNR⁷R⁸, wherein r is an integer of 2 to 5, and R⁷ and R⁸ are each independently hydrogen or C₁-C₃-alkyl, with the proviso that when X is N, R¹ is hydrogen,
R² represents hydrogen, halogen or a C₁-C₆-alkoxy group, represents —(CH₂)ₚCO₂R⁷, —(CH₂)ₚOR⁷, —(CH₂)ₚNR⁷R⁸, —NHR¹⁰, —N(H)S(O)₂R⁷ or —NHC(O)R¹⁰, or represents —(CH₂)ₚ-heterocycle-R¹⁰ which is a 5 to 6-membered ring in which the heterocycle portion contains 1 or 2 heteroatoms selected from N, O and S atoms, wherein p is an integer of 0 to 3, R⁷ and R⁸ are as defined above, and R¹⁰ represents hydrogen, oxo, C₁-C₆-alkylcarbonyl, C₁-C₆-alkoxy or C₁-C₆-alkyl or represents a 5 to 6-membered heterocycle containing 1 or 2 nitrogen atoms as a heteroatom,
R³ represents hydrogen, halogen, C₁-C₆-alkyl or phenyl, or represents —(CH₂)ₙ-heterocycle which a 5 to 6-membered ring containing 1 or 2 heteroatoms selected from N, O and S atoms wherein n is an integer of 0 to 3, with the proviso that when X is C and m is 0, R³ is phenyl, and when X is N, R³ is hydrogen or phenyl,
R⁴ represents —YR¹¹, wherein Y is a direct bond, or represents —(CR⁷R⁸)ₚY′—, wherein p is an integer of 0 to 3, R⁷ and R⁸ are as defined above,
Y′ is selected from the group consisting of —O—, —C(O)— and —C(O)O—, R¹¹ is selected from the group consisting of hydrogen, halogen, C₁-C₆-alkyl and —(CH₂)ₜB—R¹³, t is an integer of 0 to 3, B represents a 5 to 6-membered heterocycle containing 1 or 2 heteroatoms selected from N, O and S atoms, or represents C₆-C₁₀-aryl, and R¹³ represents hydrogen, cyano, halogen, hydroxy, oxo, thiol, carboxy or carboxy-C₁-C₆-alkyl, with the proviso that when X is N, R⁴ represents hydrogen or C₁-C₆-alkyl,
R⁵ represents hydrogen, C₁-C₆-alkyl, C₃-C₆-cycloalkyl, heterocycle or heterocyclyl-C₁-C₆-alkyl, wherein the heterocycle is a 3 to 8-membered ring containing 1 to 3 heteroatoms selected from N and O atoms, with the proviso that when X is N, R⁵ is hydrogen, and
R⁶ represents —(CR⁷R⁸)ₚ—Z-D-W—R¹⁴, wherein Z represents a direct bond, or is selected from the group consisting of —C(O)— and —C(O)O—, D represents a direct bond, represents C₄-C₆-cycloalkyl, represents a 5 to 6-membered heteroaryl containing 1 or 2 N atoms, or represents a 5 to 6-membered heterocycle containing 1 or 2 heteroatoms selected from N, O and S atoms, W represents a direct bond, or represents —NR⁷—, —C(O)—, —C(O)O—, —C(O)NR¹²— or —S(O)_{y}—, R¹² represents hydrogen, C₁-C₃-alkyl or C₆-C₁₀-aryl, y is an integer of 1 or 2, and R¹⁴ represents hydrogen, hydroxy, C₁-C₆-alkyl, a 5 to 6-membered heterocycle containing 1 or 3 heteroatoms selected from N, O and S atoms, or C₆-C₁₀-ar-C₁-C₆-alkyl,
with the proviso that when X is N, R⁶ represents C₄-C₆-cycloalkyl, or represents a 5 to 6-membered heterocycle containing 1 or 2 heteroatoms selected from N, O and S atoms,
wherein alkyl, alkoxy, aryl, cycloalkyl, heterocycle and heteroaryl may be optionally substituted, and the substituents are one or more selected from the group consisting of hydroxy, C₁-C₆-alkylamino, di(C₁-C₆-alkyl)amino, carboxy, C₁-C₆-alkyl, C₁-C₆-alkoxy, carboxy-C₁-C₆-alkyl and oxo.

2. The method according to claim 1, wherein the ophthalmologic disease is cataract, glaucoma, retinal degeneration, retinal pigment degeneration, retinal detachment, retinal tear, retinal ischemic disease, diabetic retinal retinopathy, keratoconjunctival epithelial damage or corneal epithelial wound.

3. The method according to claim 1, wherein
n is an integer of 1 to 3,
m is 0 or 1, with the proviso that when X is N, m is 0,
A represents phenyl,
X represents C or N,
R¹ represents hydrogen, C₁-C₆-alkyl or —(CH₂)ᵣNR⁷R⁸, r is an integer of 2 to 3, R⁷ and R⁸ are each independently hydrogen or C₁-C₃-alkyl,
R² represents hydrogen, halogen, —(CH₂)ₚCO₂R⁷, —(CH₂)ₚOR⁷, —(CH₂)ₚNR⁷R⁸, —NHR¹⁰, —N(H)S(O)₂R⁷ or —NHC(O)R¹⁰, or represents —(CH₂)ₚ-heterocycle-R¹⁰ which is a 5 to 6-membered ring containing 1 or 2 heteroatoms selected from N, O and S atoms,
p is an integer of 0 to 3,
R¹⁰ represents hydrogen, oxo, C₁-C₆-alkylcarbonyl or C₁-C₆-alkyl, or represents a 5 to 6-membered heterocycle which contains 1 to 2 nitrogen atoms as a heteroatom and is optionally substituted by C₁-C₃-alkyl,
R³ represents hydrogen, halogen or C₁-C₆-alkyl, or represents phenyl optionally substituted by C₁-C₆-alkoxy, or represents heterocyclyl-C₁-C₃-alkylene which is a 5 to 6-membered ring wherein the heterocycle contains 1 to 2 heteroatoms selected from N and O atoms and is optionally substituted by 1 or 2 oxo groups, with the proviso that when X is C and m is 0, R³ is phenyl, and when X is N, R³ is hydrogen or phenyl,
R⁴ represent —YR¹¹, wherein Y is a direct bond or —(CR⁷R⁸)ₚY′—, Y′ is selected from the group consisting of —O—, —C(O)— and —C(O)O—, R¹¹ is selected from the group consisting of hydrogen, halogen, C₁-C₆-alkyl, hydroxy-C₁-C₆-alkyl and —(CH₂)ₜB—R¹³, t is an integer of 0 to 3, B represents C₆-C₁₀-aryl, or represents a 5 to 6-membered heterocycle containing 1 to 2 heteroatoms selected from N, O and S atoms, R¹³ represents hydrogen, halogen, hydroxy, oxo, thiol, carboxy or carboxy-C₁-C₆-alkyl,
R⁵ represents hydrogen, C₁-C₆-alkyl, C₃-C₆-cycloalkyl, heterocycle or heterocyclyl-C₁-C₆-alkyl, wherein the heterocycle is a 3 to 8-membered ring that contains 1 to 3 heteroatoms selected from N and O atoms and is optionally substituted by 1 or 2 oxo groups, with the proviso that when X is C and m is 0, R³ is phenyl, and when X is N, R³ is hydrogen or phenyl, with the proviso that when X is N, R⁵ is hydrogen,
R⁶ represents —(CR⁷R⁸)ₚ—Z-D-W—R¹⁴,
Z represents a direct bond, or is selected from the group consisting of —C(O)— and —C(O)O—,
D represents C₄-C₆-cycloalkyl, or represents heterocycle is a 5 to 6-membered heterocycle that contains 1 to 2 heteroatoms selected from N, O and S atoms and optionally contains an oxo group,
W represents a direct bond, or represents —NR⁷—. —C(O)—, —C(O)O—, —C(O)NR¹²— or —S(O)_{y}—, y is an integer of 1 or 2, R¹² represents hydrogen or C₁-C₃-alkyl, and
R¹⁴ represents hydrogen, hydroxy, C₁-C₆-alkyl, hydroxy-C₁-C₆-alkyl, carboxy-C₁-C₆-alkyl or C₆-C₁₀-ar-C₁-C₆-alkyl, or represents a 5 to 6-membered heterocycle that contains 1 to 3 heteroatoms selected from N, O and S atoms and is optionally substituted by 1 or 2 oxo groups, with the proviso that when X is N, R⁶ represents C₄-C₆-cycloalkyl, or represents a 5 to 6-membered heterocycle containing 1 to 2 heteroatoms selected from N, O and S atoms.

4. The method according to claim 1, wherein the compound is a compound represented by formula (1a): 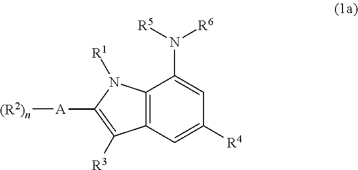
wherein n, A, R¹, R², R³, R⁴, R⁵ and R⁶ are the same as defined in claim 1.

5. The method according to claim 3, wherein R¹ is hydrogen, C₁-C₆-alkyl or di(C₁-C₃-alkyl)amino-C₂-C₃-alkyl.

6. The method according to claim 3, wherein R¹ is hydrogen, methyl or (dimethylamino)ethyl.

7. The method according to claim 3, wherein R² represents hydrogen, amino, halogen, carboxy, carboxy-C₁-C₃-alkyl, C₁-C₃-alkoxycarbonyl, C₁-C₃-alkoxycarbonyl-C₁-C₃-alkyl, hydroxy-C₁-C₃-alkyl optionally substituted by one oxo group, C₁-C₃-alkoxy, —(CH₂)ₚNR⁷R⁸, —NHR¹⁰, N(H)S(O)₂R⁷ or —NHC(O)R¹⁰ or, represents —(CH₂)ₚ-heterocycle-R¹⁰, wherein heterocycle, p, R⁷, R⁸ and R¹⁰ are the same as defined in claim 2.

8. The method according to claim 7, wherein R² is selected from the group consisting of hydrogen, methoxy, fluoro, —NH₂, —NHAc, —NHSO₂Me, —NHBOC, —NH(1-methyl-piperidine), 1-oxo-2-hydroxy-ethyl, dimethylaminomethyl, hydroxymethyl, hydroxyethyl, carboxy, carboxymethyl, carboxyethyl, —CH₂-[(2-oxo)piperazine], —CH₂-piperazine, —CH₂-morpholine, —CH₂-[1,1-dioxo-thiomorpholine-4-yl] and —CH₂-[4-acetyl-piperazine-1-yl].

9. The method according to claim 3, wherein R³ represents hydrogen, methyl or bromo, represents phenyl optionally substituted by C₁-C₃-alkoxy, or represents heterocyclyl-C₁-C₃-alkylene which is a 5 to 6-membered ring optionally substituted by 1 or 2 oxo groups in which the heterocycle contains 1 or 2 heteroatoms selected from N and O atoms.

10. The method according to claim 9, wherein R³ is selected from the group consisting of hydrogen, methyl, bromo, phenyl, 4-MeO-phenyl, —CH₂-(2-oxo-piperazine-4-yl), and —CH₂-(morpholine-4-yl).

11. The method according to claim 3, wherein R³ is selected from the group consisting of a direct bond, —O—, —C(O)—, and —CH₂C(O)—.

12. The method according to claim 3, wherein R¹¹ is selected from the group consisting of hydrogen, methyl, ethyl, phenyl, fluoro, chloro, 2-carboxy-pyrrolidine-1-yl, pyrrolidine-1-yl, 4-acetic acid-1,3-thiazolin-2-yl, —CH₂-(1,1-dioxo-thiomorpholine-4-yl) and —CH₂-(2-oxopiperazine-4-yl).

13. The method according to claim 3, wherein R⁵ represents hydrogen, C₁-C₆-alkyl, C₃-C₆-cycloalkyl, heterocycle or heterocyclyl-C₁-C₆-alkyl, wherein the heterocycle is a 5 to 6-membered ring containing 1 or 2 heteroatoms selected from N and O atoms and optionally substituted by 1 or 2 oxo groups.

14. The method according to claim 13, wherein R⁵ is selected from the group consisting of hydrogen, methyl, cyclopentyl, tetrahydropyran-4-yl and CH₂-(tetrahydropyran-4-yl).

15. The method according to claim 3, wherein D is selected from the group consisting of cyclopentyl, cyclohexyl, pyrrolidine, tetrahydropyran, tetrahydrofuran and piperidine.

16. The method according to claim 3, wherein W represents a direct bond, or represents —SO₂—, —CO—, —C(O)O— or —CONR¹²—, wherein R¹² is the same as defined in claim 3.

17. The method according to claim 16, wherein W is selected from the group consisting of —SO₂—, —CO—, —C(O)O—, —CON(Me)- and —CONH—.

18. The method according to claim 3, wherein R¹⁴ represents hydrogen, hydroxy, C₁-C₆-alkyl, hydroxy-C₁-C₆-alkyl or C₆-C₁₀-ar-C₁-C₃-alkyl, or represents a 5 to 6-membered heterocycle containing one N or O atom and optionally substituted by 1 or 2 oxo groups.

19. The method according to claim 18, wherein R¹⁴ is selected from the group consisting of hydrogen, hydroxy, methyl, ethyl, isobutyl, hydroxymethyl, hydroxyethyl, tetrahydrofuran, tetrahydropyran and 1,1-dioxo-tetrahydro-thiopyran.

20. The method according to claim 1, wherein the compound is selected from the group consisting of
Cyclopentyl-[5-methyl-2-phenyl-1H-indole-7-yl]-amine;
4-[(5-Chloro-2-phenyl-1H-indole-7-yl)amino]-cyclohexane-1-one;
7-(Cyclopentyl)amino-2-phenyl-1H-indole-5-carboxylic acid ethyl ester;
Cyclopentyl-[5-hydroxymethyl-2-phenyl-1H-indole-7-yl]-amine;
7-(Cyclopentyl)amino-2-phenyl-1H-indole-5-carboxylic acid;
2-[7-(Cyclopentyl)amino-2-phenyl-1H-indole-5-yl]-acetic acid ethyl ester;
2-[7-(Cyclopentylamino)-2-phenyl-1H-indole-5-yl]ethanol;
2-[7-(Cyclopentyl)amino-2-phenyl-1H-indole-5-yl]acetic acid;
2-[2-Phenyl-7-(tetrahydropyran-4-yl)amino-1H-indole-5-yl]-acetic acid;
2-[2-Phenyl-7-(1,1-dioxo-tetrahydro-thiopyran-4-yl)amino-1H-indole-5-yl]-acetic acid;
(Tetrahydropyran-4-yl)-[2-phenyl-5-(1,1-dioxo-thiomorpholine-4-yl)methyl-1H-indole-7-yl]amine;
(Tetrahydropyran-4-yl)-[2-phenyl-5-(2-oxo-piperazine-4-yl)methyl-1H-indole-7-yl]amine;
Cyclopentyl-[2-(3-fluoro)phenyl-5-(2-oxo-piperazine-4-yl)methyl-1H-indole-7-yl]amine;
(Tetrahydropyran-4-yl)-[2-(4-methoxyl)phenyl-5-(1,1-dioxo-thiomorpholine-4-yl)methyl-1H-indole-7-yl]amine;
Cyclopentyl-[3,5-dimethyl-2-phenyl-1H-indole-7-yl]-amine;
(Tetrahydropyran-4-yl)-(5-methyl-2-phenyl-1H-indole-7-yl)-amine;
Cyclopentylmethyl-(5-methyl-2-phenyl-1H-indole-7-yl)-amine;
(Tetrahydropyran-4-ylmethyl)-(5-methyl-2-phenyl-1H-indole-7-yl)-amine;
(1-Methylpiperidine-4-yl)-(5-methyl-2-phenyl-1H-indole-7-yl)-amine;
1-[4-[(5-Methyl-2-phenyl-1H-indole-7-yl)amino]piperidine-1-yl]ethanone;
Cyclopentyl-(5-chloro-2-phenyl-1H-indole-7-yl)-amine;
Cyclohexyl-(5-chloro-2-phenyl-1H-indole-7-yl)-amine;
(Tetrahydropyran-4-yl)-(5-chloro-2-phenyl-1H-indole-7-yl)-amine;
Cyclopentylmethyl-(5-chloro-2-phenyl-1H-indole-7-yl)-amine;
(Tetrahydropyran-4-ylmethyl)-(5-chloro-2-phenyl-1H-indole-7-yl)-amine;
(1-Benzylpyrrolidine-3-yl)-(5-chloro-2-phenyl-1H-indole-7-yl)-amine;
(1-Methylpiperidine-4-yl)-(5-chloro-2-phenyl-1H-indole-7-yl)-amine;
(1,4-Dioxaspiro[4.5]decane-8-yl)-(5-chloro-2-phenyl-1H-indole-7-yl)-amine;
2-[(5-Chloro-2-phenyl-1H-indole-7-yl)amino]propane-1,3-diol;
(Tetrahydropyran-4-yl)-(5-methyl-2-phenyl-1H-indole-7-yl)-methyl-amine;
(Tetrahydropyran-4-ylmethyl)-[2-phenyl-5-(1,1-dioxo-thiomorpholine-4-yl)methyl-1H-indole-7-yl]amine;
Di(tetrahydropyran-4-ylmethyl)-[2-phenyl-5-(1,1-dioxo-thiomorpholine-4-yl)methyl-1H-indole-7-yl]amine;
Di(tetrahydropyran-4-yl)-[2-phenyl-5-(1,1-dioxo-thiomorpholine-4-yl)methyl-1H-indole-7-yl]amine;
(1-Methyl-piperidinemethyl-4-yl)-[5-fluoro-2-phenyl-1H-indole-7-yl]amine;
2-[4-[(5-Fluoro-2-phenyl-1H-indole-7-yl)amino]piperidine-1-yl]ethanol;
[1-(Tetrahydropyran-4-yl)piperidine-4-yl]-(5-fluoro-2-phenyl-1H-indole-7-yl)amine;
(Tetrahydropyran-4-yl)-(5-phenoxy-2-phenyl-1H-indole-7-yl)-amine;
(Tetrahydropyran-4-ylmethyl)-[2-phenyl-5-(2-oxo-piperazine-4-yl)methyl-1H-indole-7-yl]amine;
(Tetrahydropyran-4-yl)-[5-chloro-1-(2-diethylaminoethyl)-2-phenyl-1H-indole-7-yl]amine;
Dimethyl-(5-chloro-1-methyl-2-phenyl-1H-indole-7-yl)amine;
(Tetrahydropyran-4-yl)-(5-chloro-1-methyl-2-phenyl-1H-indole-7-yl)-methylamine;
(Tetrahydropyran-4-yl)-(5-chloro-3-phenyl-1H-indole-7-yl)-amine;
Cyclopentyl-(5-chloro-3-phenyl-1H-indole-7-yl)-amine;
(Tetrahydropyran-4-ylmethyl)-(5-chloro-3-phenyl-1H-indole-7-yl)-amine;
Cyclopentyl-(5-chloro-3-(morpholine-4-yl)methyl-2-phenyl-1H-indole-7-yl)-amine;
(Tetrahydropyran-4-yl)-(5-chloro-3-(morpholine-4-yl)methyl-2-phenyl-1H-indole-7-yl)-amine;
(Tetrahydropyran-4-yl)-(5-chloro-3-(2-oxo-piperazine-4-yl)methyl-2-phenyl-1H-indole-7-yl)-amine;
Cyclopentyl-(5-chloro-3-(2-oxo-piperazine-4-yl)methyl-2-phenyl-1H-indole-7-yl)-amine;
(Tetrahydropyran-4-ylmethyl)-(5-chloro-3-(2-oxo-piperazine-4-yl)methyl-2-phenyl-1H-indole-7-yl)-amine;
(Tetrahydropyran-4-yl)-(3-bromo-5-(1,1-dioxo-thiomorpholine-4-yl)methyl-2-phenyl-1H-indole-7-yl)-amine;
Cyclopentyl-(3-bromo-5-(1,1-dioxo-thiomorpholine-4-yl)methyl-2-phenyl-1H-indole-7-yl)-amine;
(Tetrahydropyran-4-ylmethyl)-(3-bromo-5-(1,1-dioxo-thiomorpholine-4-yl)methyl-2-phenyl-1H-indole-7-yl)-amine;
(Tetrahydropyran-4-yl)-(5-(1,1-dioxo-thiomorpholine-4-yl)methyl-2-(3-fluorophenyl)-1H-indole-7-yl)-amine;
(Tetrahydropyran-4-yl)-(5-chloro-3-phenyl-1H-indole-7-yl)-amine;
(3-Methylbutyl)-[5-(1,1-dioxo-thiomorpholine-4-yl)methyl-2-(4-methoxyphenyl)-1H-indole-7-yl]-amine;
t-Butyl N-[4-[5-chloro-7-(cyclopentylamino)-1H-indole-2-yl]phenyl]carbamate;
Cyclopentyl-[2-(4-aminophenyl)-5-chloro-1H-indole-7-yl]-amine;
Cyclopentyl-{5-chloro-2-[4-(1-methyl-piperidine-4-yl)aminophenyl]-1H-indole-7-yl}-amine;
N-[4-(5-Chloro-7-cyclopentylamino-1H-indole-2-yl)-phenyl]-methanesulfoneamide;
Cyclopentyl-{5-(1,1-dioxo-thiomorpholine-4-yl)methyl-2-[4-(acetyl)aminophenyl]-1H-indole-7-yl}-amine;
Dicyclopentyl-{5-(1,1-dioxo-thiomorpholine-4-yl)methyl-2-[4-(acetyl)aminophenyl]-1H-indole-7-yl}-amine;
(Tetrahydropyran-4-yl)methyl-{5-(1,1-dioxo-thiomorpholine-4-yl)methyl-2-[4-(acetyl)aminophenyl]-1H-indole-7-yl}-amine;
Di(tetrahydropyran-4-yl)methyl-{5-(1,1-dioxo-thiomorpholine-4-yl)methyl-2-[4-(acetyl)aminophenyl]-1H-indole-7-yl}-amine;
(Tetrahydropyran-4-yl)-{5-(1,1-dioxo-thiomorpholine-4-yl)methyl-2-[4-(acetyl)aminophenyl]-1H-indole-7-yl}-amine;
(5-Methyl-2-phenyl-1H-indole-7-yl)-piperidine-4-yl-amine;
[1-(Methanesulfonyl)piperidine-4-yl]-(5-methyl-2-phenyl-1H-indole-7-yl)-amine;
2-Hydroxy-1-[4-(5-methyl-2-phenyl-1H-indole-7-yl)amino-piperidine-1-yl]-ethanone;
(5-Chloro-2-phenyl-1H-indole-7-yl)-piperidine-4-yl-amine;
4-(5-Chloro-2-phenyl-1H-indole-7-yl)amino-piperidine-1-yl-carboxylic acid phenylamide;
1-[4-(5-Chloro-2-phenyl-1H-indole-7-yl)amino-piperidine-1-yl]-2-dimethylamino-ethanone;
[5-(1,1-Dioxo-thiomorpholine-4-yl)methyl-2-phenyl-1H-indole-7-yl]-(piperidine-4-yl)methyl-amine;
(5-(1,1-Dioxo-thiomorpholine-4-yl)methyl-2-phenyl-1H-indole-7-yl)-(1-methanesulfonyl-piperidine-4-yl)-amine;
{4-[5-(1,1-Dioxo-thiomorpholine-4-yl)methyl-2-phenyl-1H-indole-7-yl]amino-piperidine-1-yl}-(tetrahydrofuran-2-yl)-methanone;
(5-Fluoro-2-phenyl-1H-indole-7-yl)-[1-(1,1-dioxo-tetrahydrothiopyran-4-yl)-piperidine-4-yl]-amine;
N-(5-Chloro-2-phenyl-1H-indole-7-yl)-N,N-dimethyl-cyclohexane-1,4- [symbol] amine;
N-(5-Chloro-2-phenyl-1H-indole-7-yl)-N′-methyl-cyclohexane-1,4- [symbol] amine;
4-(5-Chloro-2-phenyl-1H-indole-7-yl)amino-cyclohexane-1-carboxylic acid;
4-(5-Methyl-2-phenyl-1H-indole-7-yl)amino-cyclohexane-1-carboxylic acid;
4-(5-Chloro-2-phenyl-1H-indole-7-yl)amino-cyclohexane-1-carboxylic acid amide;
4-(5-Chloro-2-phenyl-1H-indole-7-yl)amino-cyclohexanecarboxylic acid methylamide;
2-(5-Fluoro-2-phenyl-1H-indole-7-yl)amino-acetic acid methyl ester;
2-(5-Fluoro-2-phenyl-1H-indole-7-yl)amino-acetic acid;
2-(5-Phenoxy-2-phenyl-1H-indole-7-yl)amino-acetic acid methyl ester;
2-[(5-Phenoxy-2-phenyl-1H-indole-7-yl)amino]-acetic acid;
2-[(5-Phenoxy-2-phenyl-1H-indole-7-yl)amino]-propionic acid methyl ester;
2-(5-Phenoxy-2-phenyl-1H-indole-7-yl)amino-propionic acid;
2-(5-Chloro-2-phenyl-1H-indole-7-yl)amino-propionic acid;
(5-Chloro-2-phenyl-1H-indole-7-yl)-pyridine-2-yl-amine;
(5-Chloro-2-phenyl-1H-indole-7-yl)-5-methyl-pyridine-2-yl-amine;
(5-Chloro-3-phenyl-1H-indole-7-yl)-(5-methyl-pyridine-2-yl)-amine;
(2S)-1-(7-Cyclopentylamino-2-phenyl-1H-indole-5-carbonyl)-pyrrolidine-2-carboxylic acid methyl ester;
(2S)-1-(7-Cyclopentylamino-2-phenyl-1H-indole-5-carbonyl)-pyrrolidine-2-carboxylic acid;
(2S)-1-[2-Phenyl-7-(tetrahydropyran-4-yl)amino-1H-indole-5-carbonyl]-pyrrolidine-2-carboxylic acid;
2-(7-Cyclopentylamino-2-phenyl-1H-indole-5-yl]-1-pyrrolidine-1-yl-ethanone;
Cyclopentyl-[2-phenyl-5-(2-pyrrolidine-1-yl-ethyl)-1H-indole-7-yl]-amine;
2-[(R)-2-(7-Cyclopentylamino-2-phenyl-1H-indole-5-yl)-4,5-dihydro-thiazole-4-yl]-acetic acid;
2-[(R)-2-(2-Phenyl-7-(tetrahydropyran-4-yl)methylamino-1H-indole-5-yl)-4,5-dihydro-thiazole-4-yl]acetic acid;
3-(7-Cyclopentylamino-5-chloro-1H-indole-2-yl)-benzoic acid methyl ester;
3-(7-Cyclopentylamino-5-chloro-1H-indole-2-yl)-benzoic acid;
[3-(5-Chloro-7-cyclopentylamino-1H-indole-2-yl)-phenyl]-methanol;
{3-[5-Chloro-7-(tetrahydropyran-4-yl)amino-1H-indole-2-yl]-phenyl}-methanol;
2-{3-[5-Chloro-7-(tetrahydropyran-4-ylmethyl)amino-1H-indole-2-yl]-phenyl}-acetic acid;
2-[3-(5-Chloro-7-cyclopentylamino-1-H-indole-2-yl)-phenyl]-acetic acid;
2-{3-[5-Chloro-7-(tetrahydropyran-4-yl)amino-1H-indole-2-yl]-phenyl}-acetic acid;
2-{3-[5-Chloro-7-(tetrahydropyran-4-ylmethyl)amino-1-H-indole-2-yl]-phenyl}-acetic acid methyl ester;
2-[3-(5-Chloro-7-cyclopentylamino-1H-indole-2-yl)-phenyl]-acetic acid methyl ester;
2-{3-[5-Chloro-7-(tetrahydropyran-4-yl)amino)-1H-indole-2-yl]-phenyl}-acetic acid methyl ester;
[2-(5-Chloro-7-cyclopentylamino-1H-indole-2-yl)-phenyl]-methanol;
2-(5-Chloro-7-cyclopentylamino-1H-indole-2-yl)-benzoic acid;
2-(5-Chloro-7-cyclopentylamino-1H-indole-2-yl)-benzoic acid methyl ester;
[4-(5-Chloro-7-cyclopentylamino-1H-indole-2-yl)-phenyl]-methanol;
2-{4-[5-Chloro-7-(tetrahydropyran-4-yl)amino-1H-indole-2-yl]phenyl}-ethanol;
4-(5-Chloro-7-cyclopentylamino-1H-indole-2-yl)-benzoic acid;
2-{4-[5-Chloro-7-(tetrahydropyran-4-ylmethyl)amino-1H-indole-2-yl]phenyl}-acetic acid;
2-[4-(5-Chloro-7-cyclopentylamino-1H-indole-2-yl)phenyl]-acetic acid;
2-{4-[5-Chloro-7-(tetrahydropyran-4-yl)amino-1H-indole-2-yl]phenyl}-acetic acid;
4-(5-Chloro-7-cyclopentylamino-1H-indole-2-yl)benzoic acid methyl ester;
4-(7-Cyclopentylamino-5-methyl-1H-indole-2-yl)benzoic acid methyl ester;
2-{4-[5-Chloro-7-(tetrahydropyran-4-ylmethyl)amino-1H-indole-2-yl]phenyl}-acetic acid methyl ester;
2-[4-(5-Chloro-7-cyclopentylamino-1H-indole-2-yl)phenyl]acetic acid methyl ester;
2-{4-[5-Chloro-7-(tetrahydropyran-4-yl)amino-1H-indole-2-yl]phenyl}-acetic acid methyl ester;
[5-Chloro-2-(3-dimethylaminomethylphenyl)-1H-indole-7-yl]-(tetrahydropyran-4-yl)-amine;
[5-Chloro-2-(3-morpholine-4-ylmethylphenyl)-1H-indole-7-yl]-(tetrahydropyran-4-yl)-amine;
1-{4-[3-(5-Chloro-7-tetrahydropyran-4-ylamino-1H-indole-2-yl)-benzyl]-piperazine-1-yl}-ethanone;
{5-Chloro-2-[3-(2-oxo-piperazine-4-yl)ethylphenyl]-1H-indole-7-yl}-(tetrahydropyran-4-yl)-amine;
{5-Chloro-2-[3-(1,1-dioxo-thiomorpholine-4-yl)ethylphenyl]-1H-indole-7-yl}-(tetrahydropyran-4-yl)-amine;
Cyclopentyl-(1H-indazole-7-yl)-amine;
(Tetrahydropyran-4-yl)-(1H-indazole-7-yl)-amine;
Cyclopentyl-(5-methyl-1H-indazole-7-yl)-amine;
(5-Methyl-1H-indazole-7-yl)-(tetrahydropyran-4-yl)-amine;
Cyclopentyl-[3-(4-methoxyphenyl)-1H-indazole-7-yl)-amine;
[3-(4-Methoxyphenyl)-1H-indazole-7-yl)]-(tetrahydropyran-4-yl)-amine;
[3-(4-Methoxyphenyl)-1H-indazole-7-yl)-(tetrahydropyran-4-ylmethyl)-amine;
(Tetrahydropyran-4-yl)-[5-(1,1-dioxo-thiomorpholine-4-yl)methyl-3-phenyl-2-trimethylsilyl-1H-indole-7-yl)-amine;
(Tetrahydropyran-4-yl)-[5-(1,1-dioxo-thiomorpholine-4-yl)methyl-3-phenyl-1H-indole-7-yl)-amine; and
(Tetrahydropyran-4-yl)-[3-bromo-5-(morpholine-4-yl)methyl-2-phenyl-1H-indole-7-yl]-amine.

21. The method according to claim 1, wherein the compound is a compound represented by formula (1b): 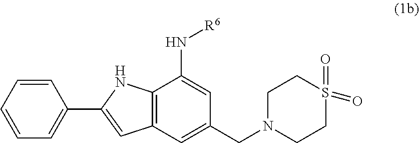
wherein
R⁶ represents —(CR⁷R⁸)ₚ—Z-D-W—R¹⁴,
R⁷ and R⁸ each independently represent hydrogen or C₁-C₃-alkyl, wherein p is an integer of 0 or 1,
Z represents a direct bond,
D represents a 5 to 6-membered heterocycle containing N or O atom,
W represents a direct bond, or represents —S(O)_{y}—, wherein y is an integer of 1 or 2, and
R¹⁴ represents hydrogen or C₁-C₆-alkyl.

22. The method according to claim 21, wherein D is tetrahydropyran or piperidine.

23. The method according to claim 21, wherein the compound is selected from the group consisting of
(Tetrahydropyran-4-yl)-[2-phenyl-5-(1,1-dioxo-thiomorpholine-4-yl)methyl-1H-indole-7-yl]amine;
(Tetrahydropyran-4-ylmethyl)-[2-phenyl-5-(1,1-dioxo-thiomorpholine-4-yl)methyl-1H-indole-7-yl]amine; and
(5-(1,1-Dioxo-thiomorpholine-4-yl)methyl-2-phenyl-1H-indole-7-yl)-(1-methanesulfonyl-piperidine-4-yl)-amine.

24. A method for cleansing or preserving a contact lens, using the compound of formula (1) according to claim 1, or a pharmaceutically acceptable salt or isomer thereof.

25. A method for preserving an artificial intraocular lens, using the compound of formula (1) according to claim 1, or a pharmaceutically acceptable salt or isomer thereof.
